(19) United States
(12) Patent Application Publication
(10) Pub. No.: US 2010/0173407 A1
(43) Pub. Date: Jul. 8, 2010

(54) INHIBITION OF GENE EXPRESSION

(75) Inventors: Krzysztof Wypijewski, Dundee Tayside (GB); Christophe Lacomme, Edinburgh Lothian (GB); Gyorgy Hutvagner, Dundee Tayside (GB); Csaba Hornyik, Dundee Tayside (GB); Jane Shaw, Tayside (GB); Jennifer Stephens, Dundee Tayside (GB)

Correspondence Address: PEPPER HAMILTON LLP, ONE MELLON CENTER, 50TH FLOOR, 500 GRANT STREET, PITTSBURGH, PA, 15219, US

(73) Assignee: SCOTTISH CROP RESEARCH INSTITUTE, Dundee Tayside (GB)

(21) Appl. No.: 12/526,612

(22) PCT Filed: Jan. 30, 2008
(86) PCT No.: PCT/GB08/50061

§ 371 Date: Oct. 27, 2009

(30) Foreign Application Priority Data
Jan. 30, 2007 (GB) 0701722.1

(51) Int. Cl. C07H 21/04; C12N 15/63; C12N 5/10; C12N 5/04

(52) U.S. Cl. 435/325; 536/23.1; 435/320.1; 435/419

(57) ABSTRACT

There is provided a method of inhibiting gene expression by locating a 5′-5 donor splicing site sequence in the 3′ UTR of a gene or within coding sequence containing the stop codon of a gene. Stability of homologous mRNA in trans is also adversely affected leading to a reduction in expression. A polynucleotide containing a 5′-donor splicing site sequence in either coding sequence containing the stop codon or the 3′ UTR is also provided and in one embodiment is in the form of a vector. The 5′-donor splicing site sequence can be present in multiple copies, for example as a tandem repeat. In one embodiment the 5-donor splicing site sequence has the sequence 5′-MAGGTRAGTA-3′ where M is A or C and R is A or G.

The present invention is concerned with a method to inhibit gene expression. In particular the method concerns inhibiting gene expression by the inclusion of a 5′-donor splicing site sequence within the 3′-untranslated region of an expressed gene or with the exon containing the stop codon of the expressed gene.

The selective regulation of gene expression is important in the control of many physiological processes. Control of gene expression could therefore allow treatment of many diseases and could also be utilised to manage infections due to pathogenic organisms such as viruses, bacteria, fungi and protozoa. Additionally, the ability to control gene expression would also have significant utility in gene discovery, particularly in determining the function of the product of a particular gene. The regulation of gene expression in cell model systems or transgenic organisms would benefit academic research as well as the commercial biotechnology industry.

One naturally occurring method of gene regulation utilises sequence specific DNA binding proteins called transcription factors. Binding of the transcription factor to its target gene results in either the activation, enhancement or inhibition of gene expression. Exemplary transcription factors include NF-kappa B, steroid hormone receptors (such as progesterone) or zinc finger proteins.

Artificial manipulation of gene expression is currently performed using antisense technology, small molecule regulators, and gene knock-outs.

Anti-sense technology is the most common approach to achieve gene-specific interference. This technique relies upon the provision of an oligonucleotide able to specifically hybridize with a portion of a target nucleic acid, which may be DNA, cDNA or, more usually, RNA. The hybridisation of the oligonucleotide to the target nucleic acid affects the ability of the target nucleic acid to replicate or undergo normal transcription (if DNA) or affects translocation within the host cell, translation, splicing or catalytic ability (if RNA). Generally, the expression or function of the target nucleic acid will be inhibited by the binding of the anti-sense oligonucleotide. The oligonucleotide selected may be a small interfering RNA (siRNA) or double-stranded RNA (dsRNA) or may alternatively be a small hairpin RNA (shRNA).

Where the target nucleotide acid is RNA, the anti-sense, shRNA or dsRNA approach is termed RNA interference (RNAi), a form of post-transcriptional gene silencing (PTGS) (Voinnet 2001, Trends Genet 17:449-459). In animal cells, RNAi technology has the disadvantage that the short oligonucleotide used to generate shRNA or dsRNA must be specific to the target nucleic acid in order to avoid unintentional interference with other genes, so that selection of suitable oligonucleotides can be difficult, and the process of selection rather laborious. Moreover, long dsRNA and its analogs trigger the interferon response and the induction of associated proteins kinases pathways, therefore masking the phenotype associated to RNAi gene knock-down.

Endogenous mechanisms associated with RNAi are believed to have evolved to protect host against RNA viral infection. Other examples of post-transcriptional control of mRNA turnover as part of the host quality control mechanism includes nonsense-mediated decay (NMD) (Hilleren and Parker 1999, Ann Rev Genet 33:229-260; Mitchell and Tollervey, 2001, Curr Opin Cell Biol 13:320-325) and the elimination of uncorrectly matured mRNA that are recognised as aberrant RNAs.

Normal gene regulation, particularly in eukaryotes, relies upon polyadenylation—the covalent linkage of 50 to 250 adenosine residues to the 3′ end of an mRNA molecule. The polyadenosine (poly-A) tail protects the mRNA from degradation by exonucleases and is also required for translation efficiency and mRNA export (L1 and Hunt, 1997, Plant Physiol 115:321-325; and Gallie, 1993, Rev Plant Physiol Plant Mol Biol 44:77-105).

Regulation of the poly-A tail addition involves a choice between several poly-A sites on a single pre-mRNA molecules, therefore generating mRNAs with different 3′ untranslated regions (3′UTR) sequences impacting on mRNA stability, localization and translatability. The 5′-donor splicing site (5′ss) sequence of Bovine papillomavirus (BPV-1) has been identified as a cis-element mediating inhibition of gene expression involved in the regulation of expression of late genes L1 and L2 coding for two capsid proteins (Furth and Baker, 1991, J Virol 65:5806-5812; Furth et al., 1994, Mol Cell Biol 14:5278-5289). In animals it is believed that a U1 small nucleolar ribonucleoprotein (U1 snRNP) particle, normally involved in recognition of the 5′ splice site during pre mRNA splicing, binds upstream of a poly-A site inhibiting its usage. This inhibitory mechanism is reminiscent of U1A autoregulation (Boelens et al., 1993, Cell 72:881-892). By this means U1A autoregulates its production by binding its own pre-mRNA and inhibiting polyadenylation (Gunderson et al, 1994, Cell 76:531-541).

So far the mechanism of inhibitory activity of a 5′ss in a 3′-UTR has been described only in mammalian cells (Furth et al., 1994, Mol Cell Biol 14:5278-5289). It was reported more recently that binding of a mutated U1 snRNA at complementary sites located within the terminal exon of pre-mRNA, directs the mRNA for degradation (Fortes et al., 2003, Proc Natl Acad Sci USA 100:8264-8269). A similar approach has been described where a modified U1snRNP harbouring in its 5′-end, a sequence complementary to the 3′-UTR led to the degradation of the target mRNA (Liu et al, 2004, Nucl Acid Res 32:1512-1517; Rowe et al., US 2003/0082149).

We have now found that 5′ss sequence when placed at the 3′-UTR could inhibit the expression of the modified gene in cis and affect the stability of mRNA in cis and of homologous mRNA in trans. Further investigations have shown that the 5′ss sequence can be placed within the open reading frame and inhibit expression.

In addition, we have found that inclusion of a 5′ss sequence within the 3′UTR of a given gene triggers degradation of the modified gene in cis and the degradation of homologous genes in trans. This mechanism consists of two distinct steps: alteration of polyadenylation status and mRNA degradation. Although the detailed mechanism of degradation is not known, the experiments reported herein clearly show that a 5′ss sequence located in an inappropriate context can function as an inhibitory element of polyadenylation and expression. The down-regulation in cis is not abolished by the p19 silencing suppressor protein suggesting that this mechanism of gene expression inhibition is distinct from other PTGS-associated pathways involving the siRNA pathway and the RNA-induced silencing complex (RISC).

U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant Mol Biol 49:77-95). Nucleotides 2-11 of the 5′ end of U1 snRNA base pair bind with the 5′ss of the pre mRNA.

We propose that interference between ribonucleoproteins binding to these 5′-splice donor sites (such as U1 snRNP) located in the vicinity of the polyadenylation signals of the RNA transcript will interfere with polyadenylation and therefore would lead to an incompletely processed, immature pre-mRNA that would be recognized as aberrant and eventually degraded.

Knock down approaches based on splicing components have been previously described (Fortes et al, 2003, Proc Natl Acad Sci USA 100:8264-8269). In that approach a modified U1snRNA (part of the U1snRNP splicing component) which carries a 10-nucleotide modified guide-sequence making it complementary to targeted mRNA, binds to the target sequence and subsequently interferes with the polyadenylation process and gene expression in mammalian cells (Rowe et al, US Patent Publication No. 2005/0043261). Such an approach is time-consuming as it requires individual U1 snRNA-based vector generation for each gene target with the risk of poor specificity due to the short guide sequence.

Splicing is a process of maturation of mRNA transcripts, which is essential for gene expression. The splicing process is common between eukaryotes (from plants to mammals) and the splicing signals are conserved (Brown and Simpson, 1998, Annu Rev Plant Physiol Plant Mol Biol 49:77-95). Moreover, as previously stated BPV-1 uses a similar mechanism of regulation to inhibit the expression of late genes in animal cells (Furth and Baker, 1991 J Virol 65:5806-5812; Furth et al., 1994, Mol Cell Biol 14:5278-5289). Therefore we predict that the 5′ss-mediated inhibition could operate both in plant and animal kingdom for high-throughput knock-down of endogenous genes. By preparing suitable expression vectors, transfection of native or transgenic GFP mammalian and C. elegans cell cultures can be achieved. Monitoring GFP expression at the protein level, mRNA level, fluorescence and its effect in trans, further targeting selected candidate genes such as collagen or osteocalcin can also be used.

The present invention therefore provides a modified polynucleotide comprising a gene having an exon containing a stop codon and said polynucleotide having a transcription terminator for said gene, wherein the gene has a sequence derived from a 5′ splicing site (hereinafter “5′ss sequence”) located upstream of the transcription terminator and within the exon containing the stop codon.

As indicated above and in example 1, it should be understood that the 5′ss sequence in each aspect of the present invention may not be involved in any splicing event. In this regard, the 5′ss sequence(s) could be considered as being “unpaired”, that is to say that the or each 5′ss sequence would not pair with a 3′ss sequence in a splicing event to excise nucleotide sequence lying between a 3′ss sequence and the 5′ss sequence.

In one embodiment, the reference to “transcription terminator” refers to the location at which transcription is terminated in vivo or in vitro using cellular extracts.

In one embodiment the 5′ss sequence is located up to and including 10000 nucleotides upstream of the transcription terminator, for example up to and including 5000 nucleotides upstream of the transcription terminator.

In one embodiment, the 5′ss sequence is located within 20 to 1000 nucleotides upstream of the transcription terminator.

Optionally the 5′ss sequence is located up to and including 50, 100, 200, 300, 400, 500, 600, 700, 800 or 900 nucleotides upstream of the transcription terminator.

In one embodiment, the modified polynucleotide comprises a polyadenylation signal downstream of the 5′ss sequence.

As indicated above, where the 5′ss sequence is located in coding sequence, it must be located within the same portion of coding sequence as the stop codon for the gene. The 5′ss can be located upstream or downstream of the stop codon. The 5′ss sequence must not be separated from a polyadenylation signal by an intron. Of course, where the gene of interest does not contain introns, there will be no such restriction on the location of the 5′ss sequence.

As used herein the term “derived from” means that the 5′ss sequence has at least 6 contiguous nucleotides copied from at least a portion of a 5′ splicing site. The 5′ss sequence can have at least 10 contiguous nucleotides copied from a 5′ splicing site.

In one embodiment, the 5′ss sequence inhibits gene expression.

In one embodiment, the 5′ss sequence is located downstream of the stop codon for the gene.

In one embodiment, more than one copy of the 5′ss sequence may be inserted. For example two or more copies of the 5′ss sequence can be inserted as repeats whether in tandem or separated by one or more nucleotides. The copies of the 5′ss sequence can be inserted as direct repeats. Optionally, three, four or five copies of the 5′ss sequence can be present. In one embodiment 6, 7, 8, 9, 10, 11, 12, 13, 14, 15, 16, 17, 18, 19 or 20 copies of the 5′ss sequence can be present. In one embodiment two 5′ss sequences are present as tandem repeats. Multiple copies of these tandem repeats can be present, for example 2, 3, 4, 5, 6, 7, 8, 9 or 10 copies of the tandem repeats can be present.

In one embodiment a spacer of from 0 to 10000 nucleotides between each 5′ss sequence can be present. Where more than two copies of the 5′ss sequence is present, each spacer can be independently selected. Where tandem repeats of two 5′ss sequences as referenced above are present, these can be spaced apart from other 5′ss sequences (optionally also in the form of tandem repeats) by a spacer.

In one embodiment, the spacer can be selected from 0 to 5000 nucleotides, for example from 0 to 1000 nucleotides. In one embodiment the spacer is from 0 to 100, for example 0 to 50 nucleotides, for example the spacer can be 0, 1, 2, 3, 4, 5, 6, 7, 8, 9 or 10 nucleotides.

The polynucleotide described above can be used to promote mRNA instability, both in cis and in trans. Where the effect is observed in trans the mRNA affected is transcribed from a gene (the target gene) having a targeted sequence which is identical or homologous to at least a portion of the gene of the polynucleotide. In one embodiment the targeted sequence is at least 21 nucleotides in length and has homology with a 21 nucleotide portion of the polynucleotide. In one embodiment “homology” can be considered as at least 90% sequence identity, for example 91%, 92%, 93%, 94%, 95%, 96%, 97%, 98% 99% or 100% sequence identity, with the portion of the polynucleotide.

The percent identity of two amino acid sequences or of two nucleic acid sequences may be determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov.

Where high degrees of sequence identity are present there will be relatively few differences in amino acid sequence. Thus for a 21 nucleotide sequence, change of a single nucleotide will result in a 95% sequence identity, whereas change in 2 nucleotides in the sequence will result in a 90% sequence identity.

The present invention further provides a polynucleotide vector having a 5′ splicing site sequence (hereinafter a “5′ss sequence”) located upstream of a transcription terminator.

The 5′ss sequence can be located in at least a portion of 3′ UTR of a gene or within at least a portion of an open reading frame (ORF) of a gene. Where the 5′ss sequence is placed in an ORF, it must not be separated from the stop codon by an intron, that is the ORF contains a stop codon.

In one embodiment the polynucleotide vector comprises a transcription initiator (promoter and/or start site). The transcription initiator will be in a functional relationship with the terminator and 5′ss sequence, i.e. will be located upstream of the 5′ss sequence. The transcription initiator will be able to permit transcription of DNA to RNA, for example in a host cell or in vitro, ie. can bind transcription factor(s) that recruit RNA polymerase which initiates transcription.

In one embodiment the polynucleotide vector includes at least one site to facilitate insertion of a targeting sequence between the transcription initiator and transcription terminator. In one embodiment the site is a restriction enzyme site. Suitable examples are well known in the art. In one embodiment the site is a site-specific recombination site. Suitable examples are known in the art.

The targeting sequence can be selected according to the intended use of the polynucleotide vector.

In one embodiment the targeting sequence is at least 21 nucleotides long.

The targeting sequence can be a portion of a gene encoding a protein, a portion of 3′UTR of a gene encoding a protein, or a portion of a gene for an untranslated RNA.

In one embodiment the polynucleotide vector of the present invention comprises, in functional relationship, a transcription initiator, a targeting sequence, and a transcription terminator, wherein said vector further comprises a 5′ss sequence upstream of the transcription terminator.

In one embodiment the polynucleotide vector of the present invention is suitable for inhibiting gene expression of a target gene in a cell.

The target gene will have a targeted sequence having homology with the targeting sequence of the polynucleotide vector according to the invention. In one embodiment, the targeted sequence comprises at least 21 (consecutive) nucleotides which have homology with the targeting sequence. In one embodiment, “homology” can be considered as at least 90% sequence identity with the targeting sequence, for example 91%, 92%, 93%, 94%, 95%, 96%, 97%, 98%, 99% or 100% sequence identity with the targeting sequence. In one embodiment the targeting sequence is at least 21 (consecutive) nucleotides.

In one embodiment, the gene targeted for inhibition of expression (the target gene) does not form part of the polynucleotide vector of the present invention. The polynucleotide vector is able to inhibit expression of the target gene in trans. Optionally the target gene is endogenous to the cell. In one embodiment the target gene is located in the genome of the cell.

In one embodiment the cell is a eukaryotic cell. For example, the cell can be an animal cell (optionally an insect or mammalian cell, such as a human or non-human cell) or can be a plant cell.

The vector can be designed to insert the 5′ss sequence within the 3′UTR of a target gene. In such an embodiment, the vector can conveniently also include restriction enzyme sites either side of the 5′ss sequence to facilitate its insertion at the required location. In an alternative embodiment the vector can include site specific recombination sites to facilitate its insertion at the required location.

The vector can be designed to insert the 5′ss sequence at a location within an open reading frame (ORF) of a target gene, wherein said location is not separated from the stop codon by an intron. In such an embodiment, the vector can conveniently also include restriction enzyme sites either side of the 5′ss sequence to facilitate its insertion at the required location. In an alternative embodiment the vector can include site specific recombination sites to facilitate its insertion at the required location.

In an alternative embodiment the present invention provides a polynucleotide vector able to insert a 5′ splicing site sequence such that expression of a target gene is reduced.

In an alternative embodiment, the vector is an expression vector and includes a full length sequence or a partial sequence of the target gene. In this embodiment, the 5′ss sequence is located within the 3′UTR and downstream of the stop codon, and upstream of the transcription terminator of the target gene. Alternatively the 5′ss sequence is located within the 3′UTR of an unrelated gene, as well as inside sequence derived from the target gene. The 5′ss sequence will normally be located upstream of the transcription terminator and downstream of the last exon containing the stop codon of the target gene (i.e. downstream of the stop codon). In one embodiment, the 5′ss sequence will be upstream of the stop codon and the transcription terminator. Optionally, the 5′ss sequences will be upstream of the transcription terminator and downstream of a coding region of a gene without a stop codon. Optionally, the 5′ss sequence will be downstream of a coding or non-coding cDNA portion of a gene, located upstream to the transcription terminator. Optionally, the 5′ss sequence will be located upstream of an uninterrupted open reading frame (ie. an open reading frame without an intron or 3′ acceptor splicing site) and a transcription terminator.

As indicated above, 2, 3, 4, 5 or more copies of the 5′ss sequence can be present. Optionally the 2 or more copies of the 5′ss sequence are arranged as direct repeats or in tandem. Optionally spacer(s) can be present between copies of the 5′ss sequences or between tandem copies of the 5′ss sequences, if present.

In one embodiment, the 5′ss sequence (whether in a vector or otherwise) is the sequence 5′-MAGGTRAGTA-3′ (SEQ ID No. 1), where M=A or C and R=A or G. Variants derived from this sequence in which four or less nucleotides have been substituted or deleted are also covered. Optionally, only one, two or three nucleotides have been substituted or deleted.

In one embodiment the 5′ss sequence (whether in a vector or otherwise) is the sequence 5′-CAGGTAAGTA-3′ (SEQ ID No. 2) or a variant thereof.

In one embodiment, the vector of the present invention can include the sequence 5′-MAGGTRAGTA-3′ (such as 5′-CAGGTAAGTA-3′) or a variant thereof as defined above, together with a polyadenylation signal. A suitable polyadenylation signal is AAUAAA.

The target gene can be any gene of interest. The target gene can be a structural gene (ie. transcribed to mRNA which is then translated to a protein or polypeptide) or can encode non-translated RNA such as tRNA miRNA or rRNA. The target gene could be a full length or partial cDNA or encode a partial protein/polypeptide. The target gene could be endogenous to the host cell or could be heterologous (foreign) to the host cell. The target gene could also have been manipulated genetically, for example to alter its expression product, for example the target gene could be a chimeric gene.

In one embodiment the gene includes a targeting sequence of 21 nucleotides able to bind specifically under stringent conditions to a targeted sequence of at least 21 nucleotides in length. The targeting sequence can be selected from translated or non-translated nucleotide sequence.

Stable hybridisation of polynucleic acids is a function of hydrogen base pairing. Hydrogen base pairing is affected by the degree to which the two polynucleotide strands in the duplex are complementary to each other and also the conditions under which hybridisation occurs. In particular salt concentration and temperature affect hybridisation. One of ordinary skill in the art would be aware that the effective melting temperature (E Tm) of the polynucleotide duplex is controlled by the formula.

ETm=81.5+16.6(log M[Na⁺])+0.41(% G+C)−0.72(% formamide)

Where hybridisation is conducted under stringent conditions, only sequences having a high degree of complementary base pairs will remain in duplex form. As used herein the term “stringent conditions” with respect to hybridisation refers to wash conditions of 0.1×SSC at 60 to 68° C. Optionally, the wash conditions can include a suitable concentration of SDS, for example 0.1% SDS.

In one embodiment the vector of the present invention includes a 3′UTR for replacing at least a portion of the 3′UTR of a target gene. The 3′UTR will comprise at least one copy of a 5′ss sequence, such as 5′-MAGGTRAGTA-3′ or a variant thereof, together with a polyadenylation signal, such as AAUAAA. In one embodiment the 3′UTR will comprise at least one copy of the 5′ss sequence 5′-CAGGTAAGTA-3′ or a variant thereof, together with a polyadenylation signal, such as AAUAAA.

In one embodiment the vector of the present invention is an expression vector comprising a gene to be expressed, wherein said gene has a 3′UTR comprising at least one copy of a 5′ss sequence (such as 5′-MAGGTRAGTA-3′ or a variant thereof) together with a polyadenylation signal, such as AAUAAA.

The vectors of the present invention can be used to transfect or transform host cells and the host cells cultured in conventional culture media according to methods known or described in the art.

Incorporation of cloned DNA into a suitable vector, transfection or transformation of host cells and selection of the transfected or transformed cells are all processes well known to those skilled in the art and numerous suitable methods are described in the literature (see, for example, Sambrook et al., Molecular Cloning: A laboratory Manual, 3^{rd} edition, Cold Spring Harbor Laboratory Press, 2001).

In one embodiment, the expression vector is for expression in plant cells.

In one embodiment, the expression vector is for expression in animal cells.

The present invention also includes host cells containing the modified polynucleotide or vector of the present invention.

The present invention further provides a method of reducing expression of a target gene, said method comprising modifying the ORF upstream of the stop codon or the 3′UTR of the target gene by inserting a 5′ss sequence therein. The 5′ss sequence will normally be located upstream of the transcription terminator. In one embodiment the 5′ss sequence is located downstream or upstream of the target gene stop codon if the sequence is intronless. The 5′ss sequence is located between the last intron and transcription terminator if the sequence lacks a stop codon. That is to say the 5′ss sequence must not be separated from a polyadenylation signal by an intron.

As indicated above, 2, 3, 4, 5 or more copies of the 5′ss sequence can be inserted. They can be arranged either as direct repeat or as multiple copies spaced apart from each other and located within suitable relative distance to the transcription terminator and poly(A) site to mediate inhibition of gene expression. By “suitable relative distance” we mean that each copy of the 5′ss sequence can be independently separated from the polyadenylation site by a distance of from 1 nucleotide up to 1200 nucleotides.

In one embodiment the 5′ss sequence can be 5′-MAGGTRAGTA-3′ (for example 5′-CAGGTAAGTA-3′) or a variant thereof.

The present invention will now be further described with reference to the following, non-limiting, examples and figures in which:

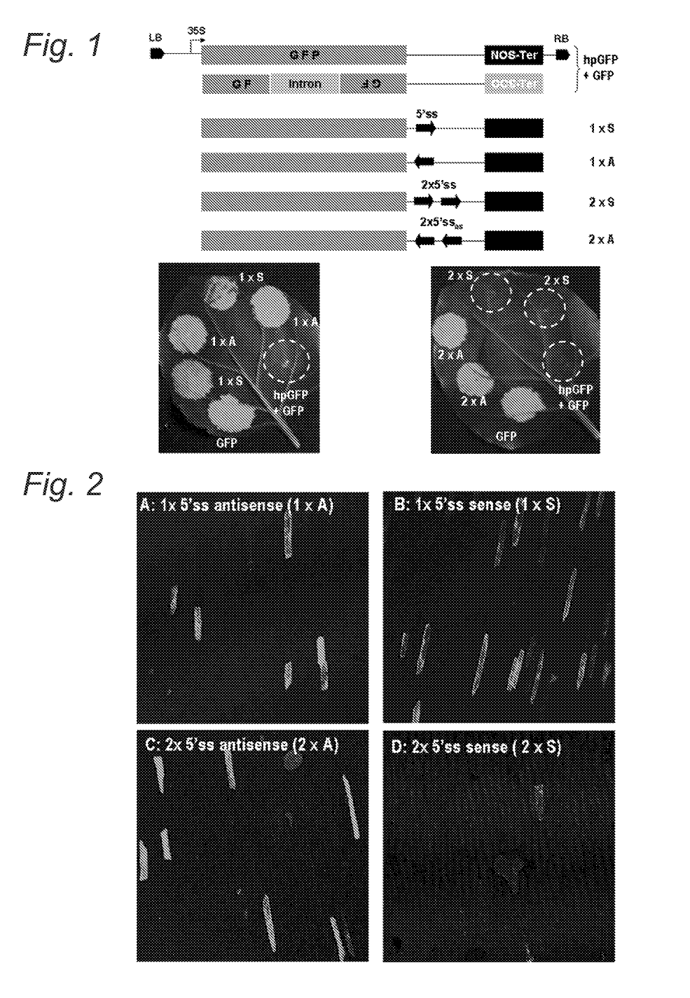

FIG. 1:

Schematic representation (not to scale) of the binary constructs and agromixes (right side) used to agroinfiltrate N. benthamiana leaves and N. benthamiana leaves observed under UV light at 3 dpi. LB, left border of the T-DNA, 35S, 35S CaMV promoter, GFP, green fluorescent protein, NOS-Ter, nopaline synthase transcription terminator, RB, right border of the T-DNA (NB: the T-DNA elements are represented only for the GFP construct and are present in all constructs used for agroinfiltration). The 5′-donor splice site is represented by an arrow in sense (1×S) or antisense orientation (1×A) or two arrows in sense (2×S) or antisense orientation (2×A).

FIG. 2:

Confocal laser scanning observation of biolistically transfected onion epidermal cells with constructs A: GFP-5′ssₐₛ (1×A), B: GFP-5′ss (1×S), C: GFP-2×5′ssₐₛ and D: GFP-2×5′ss (2×S). All images were taken at 3 dpi and at the same gain and are representative from at least three independent biolistic events (1 cm=15 μm).

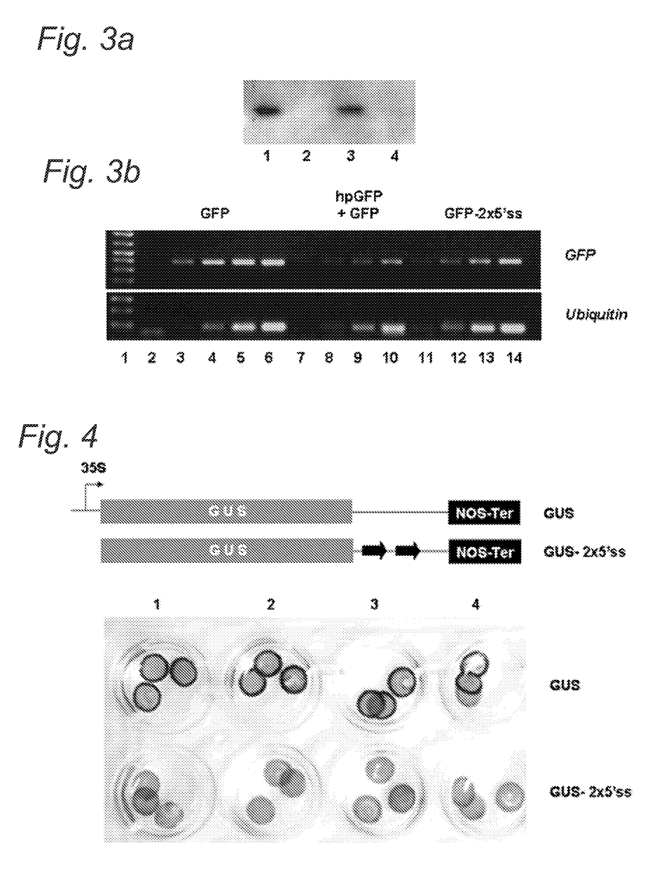

FIG. 3:

Analysis of GFP protein and mRNA levels during RNAi and 5′ss-mediated inhibition. A: western blot analysis of GFP level in leaves agroinoculated with GFP (lane 1), hpGFP+GFP (lane 2), GFP-2×5′ssₐₛ (lane 3) or GFP-2×5′ss (lane 4). Leaf discs samples were harvested and pooled from at least two different leaves from three different plants. B: Semi-quantitative RT-PCR to monitor GFP and ubiquitin mRNA levels in GFP, hpGFP+GFP, GFP-2×5′ss. Both RT-PCR products corresponding to GFP and ubiquitin mRNAs have been assessed. Lane 1, molecular weight, lane 2, non-template control, lanes 3-7-11, 22 cycles, lanes 4-8-12, 25 cycles, lanes 5-9-13, 28 cycles, lanes 6-10-14, 31 cycles. Samples were taken from two different leaves per plant from three independent plants.

FIG. 4:

GUS staining of leaves agroinfiltrated with GUS or GUS-2×5′ss constructs. A schematic representation (not to scale) of GUS and GUS-2×5′ss is presented. 3 leaf discs from two different leaves per plant from four independent plants (labelled 1, 2, 3 and 4) were excised and stained as described in material and methods. Top row: samples from leaves inoculated with GUS construct, bottom row: samples from leaves inoculated with GUS-2×5′ss construct.

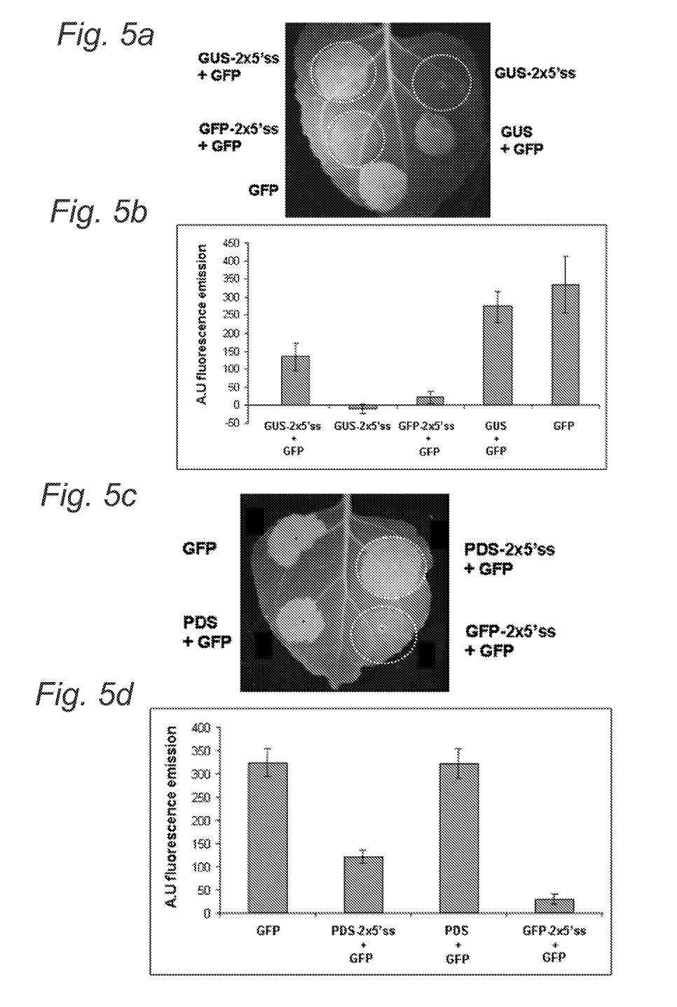

FIG. 5:

5′ss mediates inhibition of gene expression in trans.

Agroinfiltrated N. benthamiana leaves observed at 3 dpi under UV illumination. A: agroinfiltrated leaf with agromixes GUS-2×5′ss, GFP, GUS+GFP, GFP-2×5′ss+GFP, GUS-2×5′ss+GFP. B: Assessment of GFP fluorescence by spectrofluorimetry for the abovementioned agromixes, values are expressed as arbitrary units of GFP fluorescence emission as described in Material and Methods. C: agroinfiltrated leaf with agromixes PDS-2×5′ss, GFP, PDS+GFP, GFP-2×5′ss+GFP, PDS-2×5′ss+GFP. D: Assessment of GFP fluorescence by spectrofluorimetry for the abovementioned agromixes, values are expressed as Arbitrary Units (AU) of GFP fluorescence emission as described in Material and Methods.

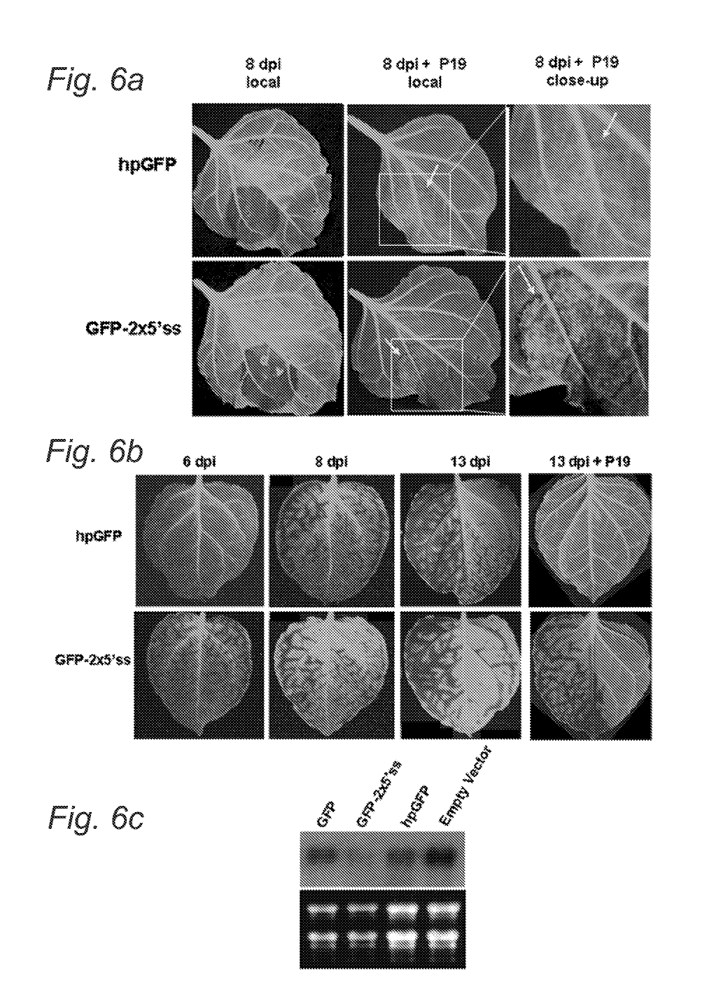

FIG. 6:

Local and systemic effect of p19 on RNAi and 5′ss-mediated inhibition. A: N. benthamiana transgenic 35S::mGFP5-ER::NOS 16c lines agroinfiltrated with hpGFP (top panels) or GFP-2×5′ss (bottom panels) in absence of p19 (left panels) or co-agroinoculated with p19 (middle and right panels) observed under UV illumination at 8 dpi. The right panels are a close-up of the boxed area from the middle panel. The arrow indicates the border of the inoculated patches appearing red for GFP-2×5′ss. B: Systemic N. benthamiana leaves observed under UV illumination at 6 dpi, 8 dpi and 13 dpi with or without p19 from 16c plants inoculated with hpGFP (upper panels) or GFP-2×5′ss (lower panels). C: Northern blot analysis of GFP mRNA levels (upper panel) of systemic leaves at 13 dpi from plants agroinoculated with GFP, GFP-2×5′ss, hpGFP and empty binary vector control. Bottom panel: UV picture of SYBR Safe staining of ribosomal RNA from the corresponding samples as a loading control.

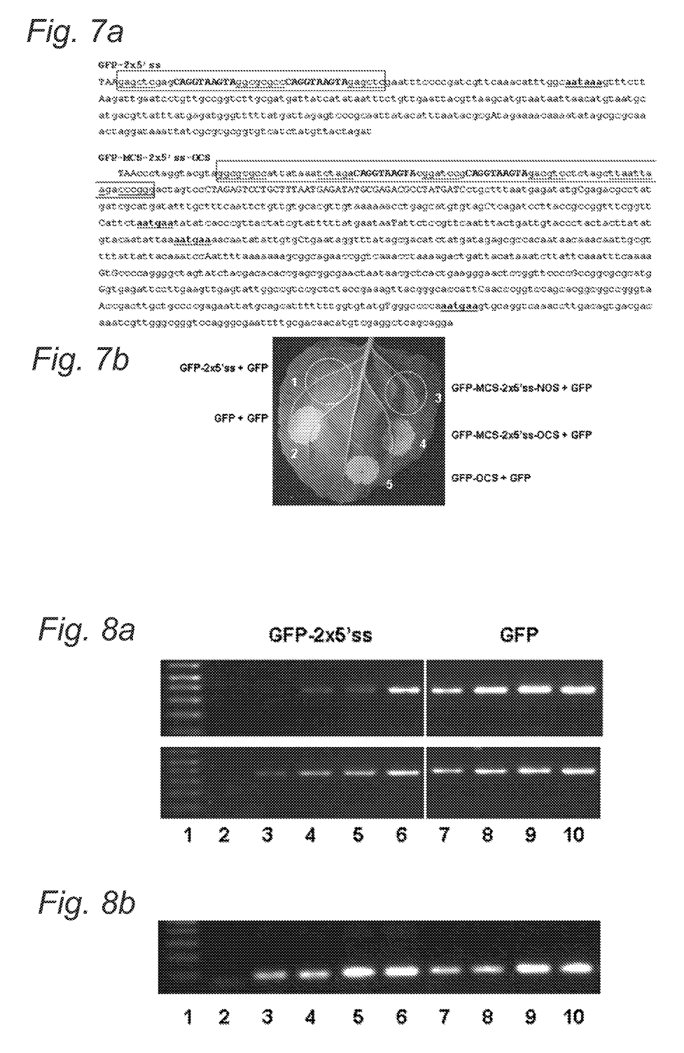

FIG. 7:

Effect of different transcriptional terminators on 5′ss-mediated inhibition. A: Nucleotidic composition of the 3′UTR of GFP-2×5′ss (SEQ ID No. 3) and GFP-MCS-2×5′ss-OCS (SEQ ID No. 4). For GFP-2×5′ss construct, the GFP stop codon TAA is upstream the inserted tandem 5′ss (in bold) including the SacI and AscI restriction sites. The 2×5′ss element is boxed. The entire NOS terminator sequence is presented downstream the 2×5′ss element with the canonical polyadenylation signal AAUAAA underlined in bold. For GFP-MCS-2×5′ss-OCS, the boxed area encompass the multiple cloning site (MCS) and the tandem repeat 2×5′ss. The entire OCS terminator sequence is presented downstream with putative polyadenylation signal AAUGAA underlined in bold. B: UV picture at 3 dpi of a N. benthamiana agroinfiltrated leaf with agromixes GFP-2×5′ss+GFP (1), GFP+GFP (2), GFP-MCS-2×5′ss-NOS+GFP (3), GFP-MCS-2×5′ss-OCS+GFP (4) and GFP-OCS+GFP (5).

FIG. 8:

Assessment of mRNA polyadenylation level in GFP-2×5′ss and GFP.

A: Semi-quantitative RT-PCR to monitor GFP mRNA levels in GFP and GFP-2×5′ss. Using first strand cDNA generated using an oligo dT primer (upper panels) or random hexamers (lower panels). Lane 1, molecular weight, lane 2, non-template control, lanes 3-7, 22 cycles, lanes 4-8, 25 cycles, lanes 5-9, 28 cycles, lanes 6-10, 31 cycles.

B: The ubiquitin mRNA levels were assessed on the same samples used for monitoring GFP mRNA levels. Ubiquitin primers (material and methods) were used to amplify the ubiquitin cDNA PCR product. Lane 1, molecular weight, lane 2, non-template control, lanes 3-6, cDNA primed with oligodT, lanes 7-10, cDNA primed with random hexamers. Lanes 3-4 and 7-8, 22 cycles, lanes 5-6 and 9-10, 25 cycles. Lanes 3, 5, 7, 9, GFP-2×5′ss, lanes 4, 6, 8, 10, GFP.

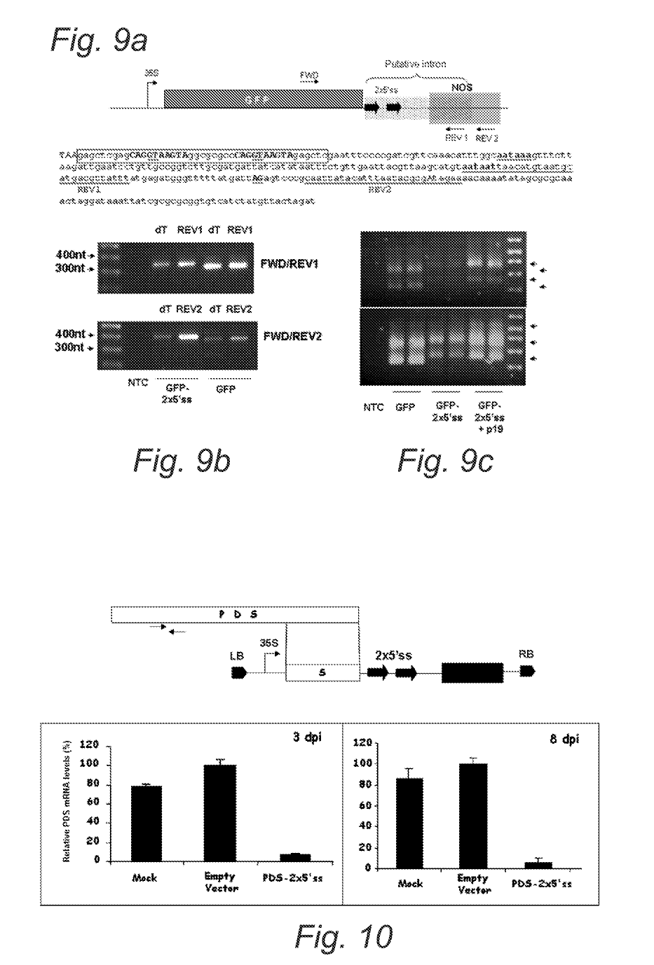

FIG. 9:

Assessment of the level of transcriptional readthrough.

A: Schematic representation (not to scale) of the GFP-2×5′ss construct with the position of 2×5′ss elements (black arrows), the NOS terminator (boxed in grey) and the position of a putative intron (dotted rectangle). The positions of the oligonucleotide primers are indicated (small arrows). The nucleotidic sequence of the 3′-UTR region (SEQ ID No. 3) is presented with the 2×5′ss element (boxed), the canonical polyadenylation site AAUAAA (bold underlined), an additional putative polyadenylation site AAUAAU (bold underlined), and a putative 3′-acceptor splicing site (AG, underlined in bold). B: Semiquantitative RT-PCR of GFP and GFP-2×5′ss of readthrough products from cDNA synthesized using oligo dT or REV1 primers and PCR amplified using FWD/REV1 primers combination (upper panel), or cDNA population synthesized using oligo dT and REV2 primers and PCR amplified using FWD/REV2 primer combination after 25 cycles of PCR amplification. C: Semiquantitative RT-PCR of GFP cDNA population amplified from samples GFP, GFP-2×5′ss and GFP-2×5′ss-Fp19 using FWD and oligo dT primer (Material and methods) at 25 and 28 cycles (upper and lower panel respectively). Ubiquitin mRNA levels were assessed similarly in these samples and showed equal amplification of PCR products for each samples (data not shown). NTC: non-template control.

FIG. 10:

Real-time RT-PCR determination of normalised relative amounts of pds mRNA levels (±SE) in PDS-2×5′ss, control empty binary vector or uninoculated N. benthamiana leaves at 3 dpi and 7 dpi. Value represents the mean from three independent plants per construct sampling two different leaves per plant (n=6). A schematic representation of N. benthamiana PDS cDNA with the position of primers used for Real-time RT-PCR analysis and the cDNA portion cloned into the binary vector is presented. LB: left border of the T-DNA, 35S: 35S CaMV promoter, white rectangle “S”: PDS cDNA fragment, tandem thick black arrows: 2×5′ss sequences, black rectangle: NOS-Ter, nopaline synthase transcription terminator, RB, right border of the T-DNA.

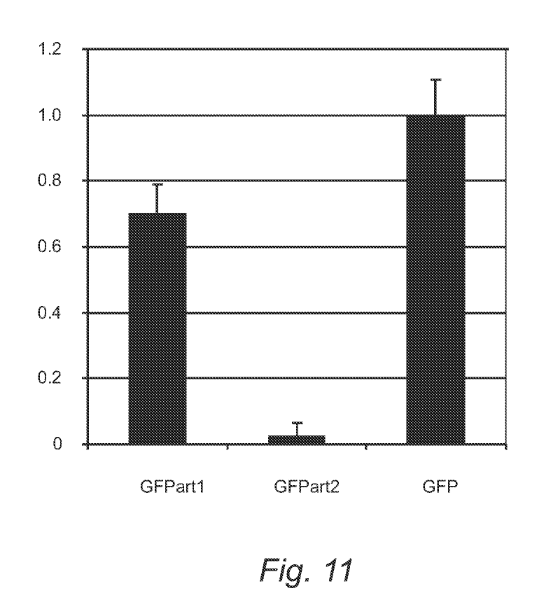

FIG. 11:

Comparison of level of gene expression down-regulation of plasmids carrying one or two impaired 5′ss located in a 3′-UTR. GFP—control; GFPart 1—1 copy 5′ss sequence; and GFPart 2—2 copies 5′ss sequence.

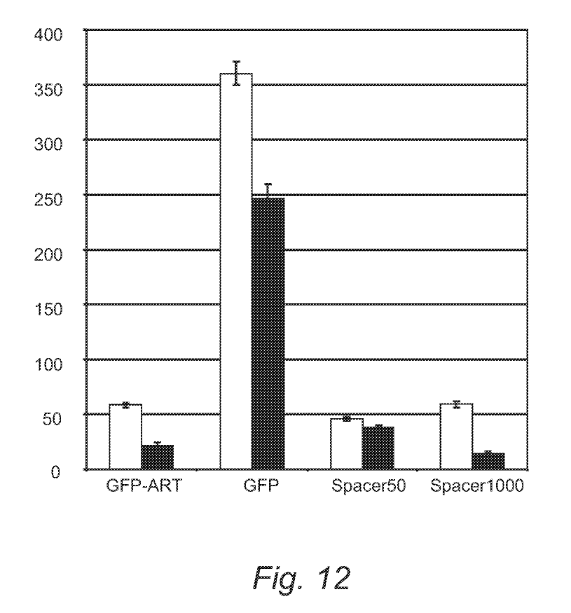

FIG. 12:

Relative fluorescence (arbitrary units) for GFP-ART (pGFP-2×5′ss), GFP (pBINmgfp5-ER), Spacer50 (pGFP-5′ss-spacer50-5′ss) and Spacer1000 (pGFP-5′ss-spacer1000-5′ss). No significant difference to downregulation observed in cis or in trans.

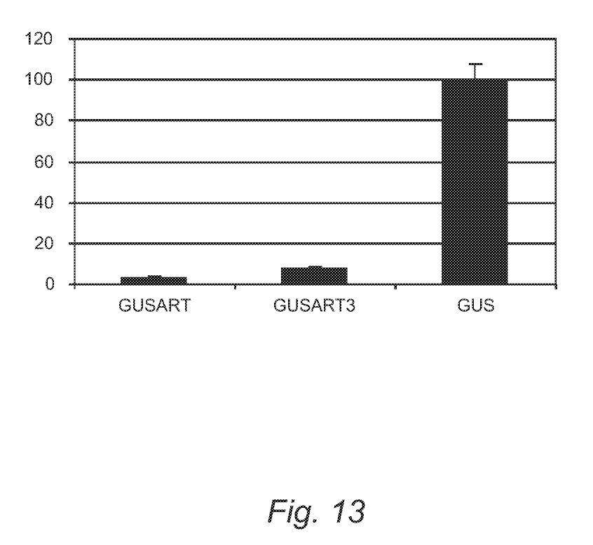

FIG. 13:

Enzymatic activity of β-glucuronidase as relative fluorescence units per minute per mg of total protein. GUSART (pGUS-2×5′ss), GUSART3 (pGUS-6×5′ss) and GUS (pBI121) (control).

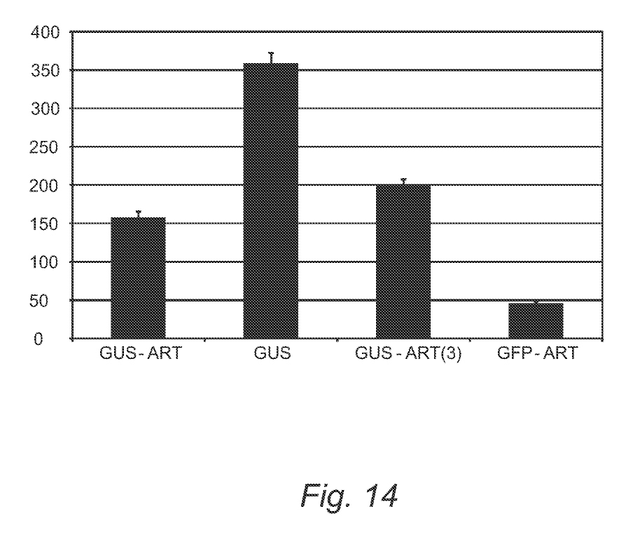

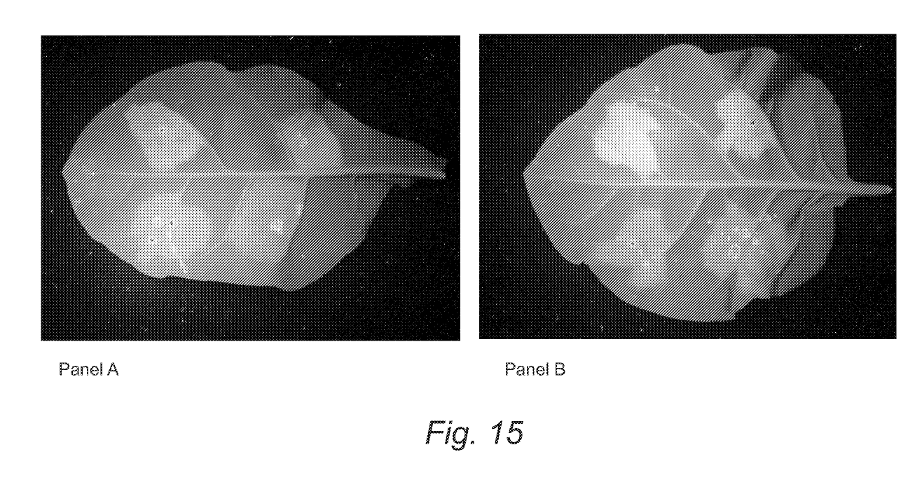

FIG. 14:

Enzymatic activity of β-glucuronidase as relative fluorescence. GUSART (pGUS-2×5′ss), GUSART(3) (pGUS-6×5′ss) and GUS (pBI121) and GFP-ART (pGFP-2×5′ss).

FIG. 15:

Agroinfiltrated Nicotiniana tabaccum leaves observed at 3 dpi under UV illumination. A: agroinfiltrated leaf with agromix GFP (pGFP-2×5′ss) in Nicotiniana tabaccum var Xanthii, B: agroinfiltrated leaf with agromix GFP (pGFP-2×5′ss) in Nicotiniana tabaccum var Samsum.

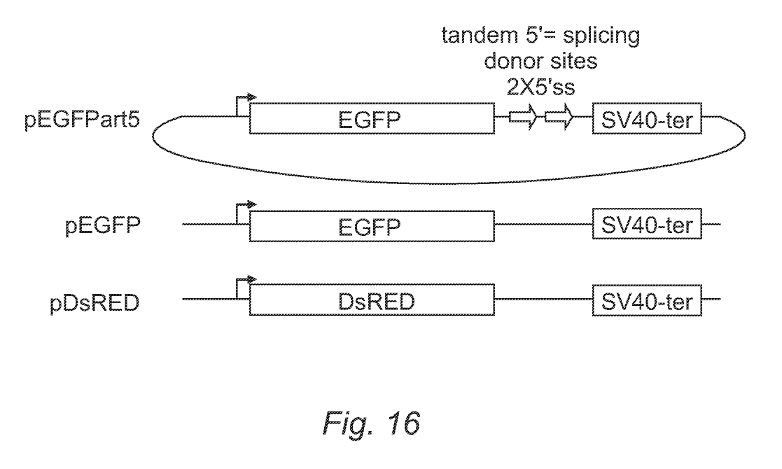

FIG. 16:

Illustrates the constructs formed as a schematic representation of pEGFP (pEGFP-C1 expression vector, Clonetech), pEGFPart5 and pDsRED constructs. As indicated above, the plasmids were constructed by inserting tandem 5′-splicing donor sites into construct pEGFP by subcloning into the mammalian expression vector pEGFP using SacI restriction site. The CMV promoter and the SV40 transcriptional terminator are represented.

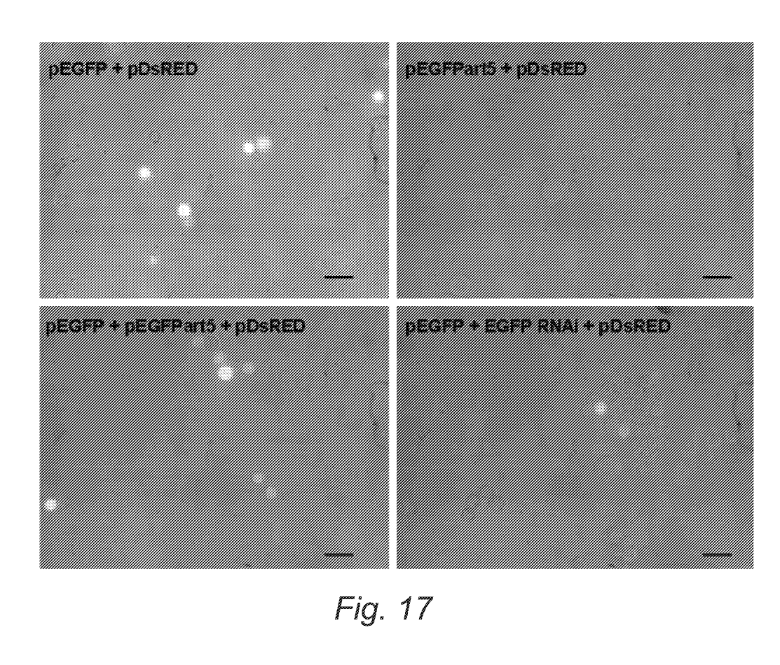

FIG. 17:

Epifluorescence and bright field merged microscopy images of HeLa cells at 2 days post transfection with the constructs pEGFP+pDsRED (upper left panel), pEGFPart5+pDsRED (upper right panel), pEGFP+pEGFPart5+pDsRED (lower left panel), pEGFP+EGFP RNAi (co-transfected siRNA EGFP)+pDsRED (lower right panel).

The scale bar represents 10 μm.

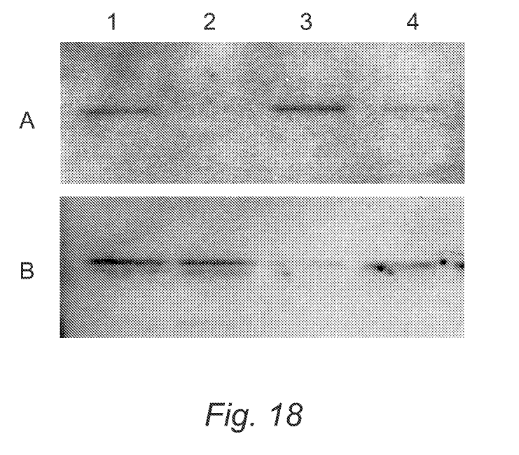

FIG. 18:

Downregulation of GFP expression by tandem insertion of 5′ss in HeLa cells. Western blot analysis shows accumulation of green fluorescence protein (panel A) or co-expressed red fluorescence protein (panel B); line 1: pEGFP+pDsRED; line 2: pEGFPart5+pDsRED; line 3: 2×pEGFP+pDsRED; line 4: pEGFP+pEGFPart5+pDsRED. The blots were probed with antibodies against green fluorescent protein (GFP) (panel A) or against red fluorescent protein (RFP) used as an internal calibrator (panel B).

### EXAMPLES

The inventors have designated the present invention as “Aberrant RNA Technology” or “ART” and references to constructs described in the examples are to be construed accordingly

To evaluate the applicability as a gene knock-down approach and study the molecular basis of this phenomenon we introduced a consensus 5′ss within the 3′-UTR of a reporter gene encoding the green fluorescent protein (GFP) and analysed the effect on gene expression transiently expressed in single cells and in plant leaf tissues.

### Example 1

### 5′ss Sequence in 3′ UTR of GFP

To evaluate the applicability as a gene knock-down approach and study the molecular basis of this phenomenon we introduced a consensus 5′ss within the 3′-UTR of a reporter gene encoding the green fluorescent protein (GFP) and analysed the effect on gene expression transiently expressed in single cells and in plant leaf tissues.

### Material and Methods:

### Agroinfiltration

All T-DNA constructs were introduced into A. tumefaciens LBA 4404 (VirG) strain by electroporation as previously described (Koscianska et al, 2003, Plant Mol Biol 59:647-661). Agrobacteria were grown overnight in LBG medium supplemented with Kanamycin (50 μg/ml) and Chloramphenicol (75 μg/ml). OD₆₀₀ was adjusted to 0.1 by diluting the bacteria in 10 mM MES, pH=5.6, 10 mM MgCl₂, 150 μM Acetosyringone and incubated for at least 1 hour at room temperature. Infiltration of the diluted bacteria was done as previously described (Johansen and Carrington, 2001, Plant Physiol 126:930-938). Plants were kept in constant conditions in growth chamber at 22° C. with a 16 hour photoperiod, light intensity ranging from 400 to 1000 μmol.m⁻² sec⁻¹. GFP fluorescence was monitored under UV illumination as previously described (Lacomme and Santa Cruz, 1999, Proc Natl Acad Sci USA 96:7956-7961). Pictures were taken using an Olympus C-2500L digital camera.

### RT-PCR and Quantification of Gene Expression

First strand cDNAs were generated using oligo-dT or random hexamers oligonucleotides (Qiagen, UK) as previously described (Lacomme et al., 2003, Plant J 34:543-553).

Semi-quantitative RT-PCR, Real-time “Taqman” RT-PCR and statistical analysis were as previously described (Lacomme et al., 2003, Plant J 34:543-553). Forward and reverse primers used for semi quantitative RT-PCR of GFP expression were 5′-GGGCACAAATTTTCTGTCAG-3′ (SEQ ID No: 5) and 5′-GTTGTGGGAGTTGTAGTTGTATTC-3′ (SEQ ID No: 6). Taqman primers for ubiquitin and phytoene desaturase (pds) were previously reported (Lacomme et al., 2003, Plant J 34:543-553). The assessment of transcriptional read-through within the NOS terminator was performed on first strand cDNA synthesized using either oligo-dT, REV1 (5′-AAATAACGTCATGCATTACATGTTAATTATT-3′) (SEQ ID No: 7), or REV2 (5′-TTCTATCGCGTATTAAATGTATAATTG-3′) (SEQ ID No: 8) primers the latest located downstream the putative 3′-acceptor splicing site and the second putative polyadenylation site AAUAAU as indicated in FIG. 9. Assessment of the length of polyadenylated GFP mRNAs was performed using oligo-dT primed cDNA further amplified using 5′-TCCACACAATCTGCCCTTTC-3′ (SEQ ID No: 9) and 5′-GCGAGCTCCGCGGCCTTTTTTTTTTTT-3′ (SEQ ID No: 10) respectively as forward and reverse primer.

### Plasmid Constructs

The plasmids used for microprojectile bombardment of onion epidermal cells were generated as follow. An expression cassette consisting of CaMV 35S::mgfp5-ER::3′-NOS derived from pBINmgfp5-ER plasmid (provided by Jim Hasselhoff et al., 1997, Proc Natl Acad Sci USA 94:2122-2127) was ampified by PCR using primers 5′-CCCAAGCTTTTTCAGAAAGAATGCTAACCC-3′ (SEQ ID No: 11) and 5′-CCCAAGCTTGATCTAGTAACATAGATGACACC-3′ (SEQ ID No: 12), digested by HindIII and cloned into a modified pBluescript KS⁺(Stratagene) from which the SacI site was removed giving pSgfpex. Into the SacI linearised pSgfpex vector annealed U1-1 or U1-2 DNA fragments generated by self-annealing of primers 5′-CGAGMAGGTRAGTAGGCGCGCCGAGCT-3′ (SEQ ID No: 13) and 5′-CGGCGCGCCTACTTACCTGCTCGAGCT-3′ (SEQ ID No: 14) for U1-1 and 5′-CGAGMAGGTRAGTAGGCGCGCCMAGGTRAGTAGAGCT-3′ (SEQ ID No: 15) and 5′-CTACTTACCTGGGCGCGCCTACTTACCTGCTCGAGCT-3′ (SEQ ID No: 16) (M is A or C; R is A or G) were ligated giving four plasmids: pSgfpU1-1s (U1-1 pair of oligos in sense orientation, further referred to as 1×S), pSgfpU1-1a (U1-1 in antisense, further referred to as 1×A), pSgfpU1-2s (U1-2 in sense, further referred to as 2×S), and pSgfpU1-2a (U1-2 in antisense, further referred to as 2×A). The plasmids used for agroinfiltration of leaves were generated as follow. Into the SacI linearised plasmid pBINmgfp5-ER. pBinmGFP5-ER sequence is deposited in GenBank (accession number 1848288). GFP insert was cloned into BamHI and SacI restriction sites of pBIN121 vector (GenBank accession number 19569229). After removing the GUS insert from pBin121 by BamHI-SacI digestion, annealed U1-1 or U1-2 pair of primers were ligated giving four plasmids: GFP-5′ss (U1-1 pair of oligos in sense orientation), GFP-5′ssₐₛ (U1-1 in antisense orientation), GFP-2×5′ss (U1-2 in sense orientation), and GFP-2×5′ssₐₛ (U1-2 in antisense orientation). Construction of GFP-MCS-2×5′ss-OCS: the GFP ORF from pBINmgfp5-ER plasmid was amplified by PCR using primers 5′-gtgtgtCTCGAGCCatgGCCAAGACTAATCTTTTTCTCTTTCTCA-3′ (SEQ ID No: 17) and 5′-gtgtgtGGCGCGCCTACGTACCTAGGgttaaccAAGCTCATCATGTTTGTAT AGTTC-3′ (SEQ ID No: 18), digested by XhoI and AscI and cloned into pFGC5941 (provided by Rich Jorgensen, University of Arizona, USA, GenBank accession number 32265027), resulting in plasmid pKWBi51. Then ChSA intron sequences were excised by AscI and PacI and ligated with annealed oligonucleotide pair 5′-CGCGCCatta taaaTCTAGACAGGTAagtaCggatccGCAGGTAagtaGACGTCctctAGCC-3′ (SEQ ID No: 19) and 5′-CCGGGGCTagagGACGTCtacttacctgCggatcc GtacttacctgTCTAGAtttataatGG-3′ (SEQ ID No: 20) and blunted by T4 DNA polymerase giving GFP-MCS-2×5′ss-OCS. The construct GFP-MCS-2×5′ss-NOS was generated by subcloning of the blunted XhoI-PacI GFP-MCS-2×5′ss from the GFP-MCS-2×5′ss-OCS construct into the XhoI-SacI blunted pBin19 vector and PCR-screened and verified by sequencing for the correct orientation. The constructs hpGFP and GFP were generated as previously described (Koscianska et al., 2005, Plant Mol Biol 59:647-661). The construct PDS-2×5′ss was generated by subcloning into the BamHI-SacI digested pBin19 binary vector the 314-bp PCR-amplified PDS cDNA fragment using oligonucleotides 5′-ATGGGATCCATGAAGGAACTAGCGAAGCTTTTC-3′ (SEQ ID No: 21) and 5′-TACGAGCTCTTAGTTCACTATGCTAACTACGCTTG-3′ (SEQ ID No: 22) (respectively forward and reverse primers) and digested by BamHI-SacI. The annealed U1-2 oligonucleotides were cloned into the SacI site of the pBin PDS construct.

### Fluorescence Spectroscopy

The green fluorescence emission in the leaf tissue was measured using a spectrofluorimeter (Spectramax M5, Molecular Devices, Excitation wavelength: 468 nm, Emission wavelength: 503 nm, Cut-off: 495 nm). Leaf discs were excised, and placed into a 96-well plate. Background autofluorescence of leaf tissue was deduced from total fluorescence. Background autofluorescence of leaf tissue was deduced from total fluorescence giving an Arbitrary Units (AU) of fluorescence emission. Microsoft Excel Software was used to perform statistical analyses and graphical presentation. All data are expressed as means±SD. Each group analysed consisted of three to eight independent samples, each sample measured from 5 to 9 independent leaf discs. All experiments were repeated at least twice.

### Histochemical Staining Procedure

Leaf discs of agroinfiltrated areas were harvested after 3 dpi. Histochemical staining for β-glucuronidase activity was performed as previously described (Jefferson et al, 1987, EMBO J 6:3901-3907; Swoboda et al., 1994, EMBO J 13:484-489).

### Micro Projectile Bombardment of Onion Epidermal Cells

Particle bombardment assays were performed on the abaxial side of adaxial epidermal peels from onion bulb scales. The method was essentially as described by Gal-On et al. (1997, Journal Virol Meth 64:103-110) and Haupt et al. (2005, Plant Cell 17:164-181). All experiments were conducted with a home-made biolistics system (Gal-On et al, 1997, Journal Virol Meth 64:103-110). Approximately 2 μg of plasmid DNA was mixed with 1 mg of tungsten particles (M-25, DuPont no. 75056) in aqueous suspension and added 70 μl of 2.5M CaCl₂ and 30 μl of 0.1M spermidine. The particles were mixed by shaking 30 min at +4° C., spun down 2 min at 10,000 g and washed with 96% ethanol. The pellet was resuspended in 45 μl of 96% ethanol. 10 μl of DNA/tungsten mixture was loaded onto the grid of a discharge assembly and left until the ethanol evaporated. Two bombardments were made before reloading the grid. Bombarded epidermis was observed under the confocal laser scanning microscope (Leica Microsystems, Heidelberg, Germany) after 3 days.

### RNA Extraction

Total RNA was extracted from 100 mg of leaf tissue from patch assay as well as from systemically silenced tissues using RNEasy kit (Qiagen, UK), TRI RNA Isolation Reagent (Sigma-Aldrich, UK) or TRIzol Reagents (Invitrogen, UK) according to the manufacturers.

The purified total RNA was used for Northern blot analysis and first strand cDNA synthesis.

### Northern Blot Analysis

Analysis of higher molecular weight RNAs was performed by Northern blot hybridization as described previously (Dalmay et al., 1993, Virology 194:697-704). Six μg of total RNA was used for separation in formaldehyde containing denaturing gel. Random priming DNA probe of GFP construct was used for detecting the high molecular weight RNAs. After hybridization signals were detected by X-ray film or phosphorimager screen visualized by FLA-7000 Fluorescent Image Analyzing System (Fujifilm).

### Results and Discussion

Insertion of Duplicated 5′ Splicing Site within the 3′-UTR of a cDNA Inhibits its Expression.

Delivery of DNA into the plant cells by agroinfiltration has been used for the transient expression of genes in plants and the induction of gene silencing (Johansen and Carrington, 2001, Plant Physiol 126:930-938). Chimeric construct GFP-5′ss-NOS harbouring a single 5′-ss in the 3′-UTR followed by the nopaline synthase (NOS) terminator was engineered and expressed in plant leaf using an Agrobacterium infiltration assay. By three days post infiltration a bright GFP fluorescence comparable to the GFP-NOS construct (FIG. 1, 1×S) was observed indicating that no significant effect on GFP expression was occurring. A construct harbouring a tandem insertion of the 5′-ss at the same location (construct GFP-2×5′ss-NOS) did not display GFP fluorescence (FIG. 1, 2×S). This behaviour was comparable to what was observed with the RNAi assay where a GFP construct was co-infiltrated with a hpGFP construct generating GFP dsRNA (FIG. 1, agromix hpGFP+GFP). Insertion of the same tandem 5′-ss in antisense orientation (FIG. 1, 2×A) did not significantly affect GFP fluorescence and was comparable to the GFP-NOS construct suggesting a strong effect of sequence polarity in triggering inhibition of GFP fluorescence. Moreover, transient expression in biolistically transfected single epidermal onion cells triggered a similar down-regulation (FIG. 2D) demonstrating that 5′-ss mediated down-regulation is observed in a different transient expression system using a different mode of DNA delivery. Assessment of GFP accumulation both at the protein and mRNA levels confirmed that GFP mRNA accumulation was strongly reduced in both GFP-2×5′ss and hpGFP in comparison to GFP or GFP-2×5′ssₐₛ samples (FIGS. 3 A and B). This demonstrates that 5′ss-mediated down-regulation is mediated by the duplicated 5′ss located within the 3′-UTR. Expression of chimeric constructs containing the bacterial uidA gene, which encodes the β-glucuronidase (GUS) reporter gene (GUS-2×5′ss-NOS), resulted in the absence of GUS staining in comparison to leaf discs infiltrated with the GUS construct (FIG. 4). This shows that GUS expression was strongly affected in the GUS-2×5′ss-NOS construct. This suggests that the 5′ss-mediated inhibition of gene expression of mRNAs encoding distinct reporter genes (GFP or GUS) is affected in a non-sequence specific manner.

5′Splicing Site Mediates Inhibition of Gene Expression in trans

The previous results suggest that the modified genes are undergoing mRNA degradation probably by being identified as aberrant and recruited by the endogenous mRNA surveillance machinery for degradation. We further investigated whether the 5′ss could mediate down-regulation of gene expression in trans.

For this purpose we co-infiltrated the constructs; GFP-2×5′ss, GUS-2×5′ss, PDS-2×5′ss (phytoene desaturase or PDS), or hpGFP with GFP, and assessed GFP fluorescence by spectrofluorimetry. All constructs harbour a NOS terminator with the exception of hpGFP where the transcription terminator originates from the octopine synthase (OCS) gene. Co-infiltration of GFP-2×5′ss and GFP resulted in inhibition of both gene expression with a 7-10-fold reduction in fluorescence emission (FIGS. 5 A and B). A similar effect was observed when GUS-2×5′ss and PDS-2×5′ss were co-infiltrated with GFP. Despite the limited sequence homology between them encompassing only the NOS terminator (FIG. 5 A to D), an approximate 2-fold decrease in GFP fluorescence was observed. It suggests, that the level of a gene expression inhibition in trans is proportional to the length of homologous sequences between the trigger and the target gene. Silencing can be triggered by non-coding sequence including transcription termination elements such as the NOS terminator (Canto et al, 2002, Molec Plant-Microbe Interact 15:1137-1146). In order to rule out any silencing effect due to the presence of the NOS terminator sequence, co-infiltration of GUS or PDS constructs with GFP were analysed. No significant GFP inhibition in trans was observed when GFP and GUS or GFP and PDS were coinoculated (FIG. 5 A to D). This confirms that 5′ss-mediated inhibition triggers a strong inhibition in trans. This suggests that stretches of sequence homology limited to the transcription terminator element are sufficient to trigger inhibition of gene expression in trans during 5′ss-mediated knock-down.

5′ss-Mediated Systemic Inhibition of Gene Expression is not Abolished by Virus-Encoded Silencing Suppressor

Plant viruses encode suppressors of silencing as a part of their counter-defense strategy to suppress host RNA-mediated defense mechanisms (Voinnet et al, 1999, Proc Natl Acad Sci USA 96:14147-14152). The protein p19 from Cymbidium ringspot virus (CymRSV) is a potent silencing suppressor that acts by sequestering siRNA and therefore preventing their incorporation into the RISC complex (Silhavy et al, 2002, EMBO J 21:3070-3080; Lakatos et al., 2004, EMBO J 23:876-884). We investigated the effect of p19-silencing suppression on 5′ss-mediated gene expression inhibition.

In the first instance we challenged N. benthamiana line 16c, which carries a highly expressed GFP transgene (Voinnet et al., 2001, Trends Genet 17:449-459), with constructs hpGFP and GFP-2×5′ss co-infiltrated with or without p19. At 8 days post inoculation in the absence of p19, the inoculated tissue had turned red due to silencing of the transgene when observed under UV illumination (FIG. 6A). In the presence of p19, the patch inoculated with the hpGFP was fluorescing brightly under UV light, indicating that silencing was suppressed (FIG. 6A, middle upper panel). For GFP-2×5′ss, an incomplete suppression of inhibition was observed and appeared as a thin border of GFP-silenced cells at the margin of agro-infiltrated zones (FIG. 6A, middle lower panel). Further, non cell autonomous RNA silencing initiated by a dsRNA construct (hpGFP) could be observed in the 16c line where a systemic inhibition of GFP fluorescence occurred by 8 dpi as shown in FIG. 6B and as previously reported (Voinnet, 2001, Trends Genet 17:449-459). GFP-2×5′ss triggers a faster systemic GFP down-regulation that was obvious by 6 dpi, preceding the systemic silencing triggered by hpGFP (FIG. 6B). When hpGFP and GFP-2×5′ss were co-infiltrated with p19, systemic inhibition of GFP expression was observed only on 16c plants co-inoculated with GFP-2×5′ss in a comparable fashion to plants inoculated only with GFP-2×5′ss (FIG. 6B). A complete suppression of GFP silencing by p19 was observed for hpGFP (FIG. 6B) as previously reported (Silhavy et al, 2002, EMBO J 21:3070-3080). Northern blot analysis of systemic leaves of 16c plants taken at the same time point after challenge with GFP, hpGFP, GFP-2×5′ss and empty vector revealed that the GFP mRNA accumulated to a lower level in the case of GFP-2×5′ss in comparison to GFP or hpGFP (FIG. 6C). This demonstrates that 5′ss-mediated inhibition is observed not only on a co-delivered transgene but also on a stably expressed transgene without being suppressed by viral-encoded silencing suppressor.

### 5′-ss Mediated Inhibition of Gene Expression is Influenced by the Nature of the Transcription Terminator and Requires an AAUAAA Polyadenylation Site.

Further mapping of the required genetic elements for GFP-2×5′ss inhibition were effectuated. A previous report has demonstrated that in a mammalian system, the presence of a canonical polyadenylation signal (AAUAAA) is required for mRNA inhibition of expression (Fortes et al, 2003, Proc Natl Acad Sci USA 100:8264-8269).

Chimeric constructs harbouring the duplicated 5′-ss were introduced upstream of the octopine synthase (OCS) terminator (FIG. 7A). The OCS terminator is widely used in plant expression systems and does not contain the AAUAAA canonical polyadenylation signal which is present within the NOS terminator. The OCS terminator harbours instead three non-canonical polyadenylation signals (AAUGAA) (FIG. 7A). When transiently expressed in agro-infiltrated leaves the construct GFP-MCS-2×5′ss-OCS displayed bright fluorescence indicating that no significant effect on gene expression was observed as opposed to the GFP-2×5′ss-NOS (FIG. 7B). This suggests that the duplicated 5′-ss element together with cis-elements from the terminator sequence are required to mediate inhibition of gene expression. The introduction of a multiple cloning site during the engineering of the duplicated 5′ss upstream of the OCS terminator (FIG. 7A, construct GFP-MCS-2×5′ss-OCS), resulted in a predicted secondary RNA structure (Brodsky et al., 1992 Dimacs 8:127-139; Brodsky et al., 1995, Biochemistry 60(8):923-928 folding as a hairpin. To investigate whether this structure had the potential to counteract the effect of 5′ss, we re-introduced the GFP-MCS-2×5′ss upstream of the NOS terminator (construct GFP-MCS-2×5′ss-NOS). Inhibition of GFP expression in cis and in trans was restored (FIG. 7B) in a similar fashion to that observed with the original GFP-2×5′ss construct. This confirmed the relative flexibility of nucleotidic sequence composition in the vicinity of the tandem 5′-ss repeat and the NOS terminator. This demonstrates that the inhibitory effect was not affected by the insertion of additional nucleotides, which could form putative RNA secondary structures. This suggests that in plants, mechanisms of mRNA expression inhibition are operating in a comparable fashion to that in mammalian cells and suggests that in plants the proximity of a AAUAAA polyadenylation signal is required to mediate 5′ss mRNA degradation.

Alteration of Polyadenylation Status is Observed in GFP-2×5′ss mRNA

As the insertion of a 5′ss is likely to affect mRNA maturation and more specifically mRNA polyadenylation, the polyadenylation status of GFP-2×5′ss mRNA was analysed. The ratio of polyadenylated and non-polyadenylated mRNA fractions for GFP and GFP-2×5′ss were determined using semi-quantitative RT-PCR from agro-infiltrated leaf tissues (FIG. 8).

Firstly, cDNA was generated for both GFP and GFP-2×5′ss samples using oligodT or random hexamers primers, each allowing the synthesis of poly(A)⁺and both poly(A)⁺and poly(A)⁻ respectively. As presented in FIG. 8A, a lower amount of poly(A)⁺mRNA is observed for GFP-2×5′ss in comparison to the GFP construct. However, by priming with random hexamers the signal of total mRNA was comparable to poly(A)⁺for GFP (FIG. 8A, lower panels) indicating that most of the GFP mRNA are correctly polyadenylated. This was not the case for GFP-2×5′ss where a higher amount of RT-PCR product was amplified if cDNA was synthesized using random hexamers primers (FIG. 8A, bottom panels) suggesting that the ratio of poly(A)⁺/total mRNA is lower for GFP-2×5′ss indicative of a deficiency in polyadenylation. In all cases a similar level of ubiquitin mRNA was detected (FIG. 8B).

Regulation of poly(A) tail addition typically involves the choice between two or more poly(A) sites on a single pre-mRNA resulting in mRNAs differing in their 3′UTR sequences. Sequence analysis of the 3′UTR of randomly selected cDNA clones from GFP and GFP-2×5′ss constructs revealed that the poly(A) tail is added at two different regions located either after the first AAUAAA poly(A) signal or after another downstream putative poly(A) signal AAUAAU (data not shown). Oligonucleotide primers for first strand cDNA synthesis and RT-PCR were designed in order to synthesize both polyadenylated and unpolyadenylated mRNA in order to discriminate between mRNAs that use the first (REV1 primer) or the second (REV2 primer) putative poly(A) signal (FIG. 9A).

As shown in FIG. 8B, poly(A) mRNA are accumulating at a lower level in GFP-2×5′ss as previously observed (FIG. 9B). A similar level of PCR product was obtained for the GFP construct for poly(A) and REV1-primed cDNA (FIG. 9B, upper panel) indicating that the first polyadenylation signal is preferentially used. A comparable level of poly(A) and REV1-primed cDNA was obtained for GFP-2×5′ss indicating that to some extent polyadenylation is still occurring in GFP-2×5′ss (FIG. 9B, upper panel). Using the downstream primer REV2 for priming, cDNA synthesis resulted in a significantly higher amplification level for GFP-2×5′ss in comparison to poly(A) fraction (FIG. 9B, lower panel). The PCR product signals obtained for GFP were much lower and of comparable intensity for the poly(A) and the REV2-primed fractions (FIG. 9B, lower panel). Taken together this suggests that GFP-2×5′ss generates a higher proportion of unpolyadenylated mRNA most likely by interfering with the poly(A) signal AAUAAA and therefore generating a higher proportion of longer mRNAs. The alternative use of the second putative poly(A) signal occurs to a much lower extent with the GFP construct (FIG. 9B). This was confirmed in FIG. 9C using oligodT and FWD primers for RT PCR. In the GFP-2×5′ss+p19 sample a greater proportion of the highest vs smallest molecular weight mRNA was obtained as opposed to GFP.

### The Inhibitory Effect Mediated by 5′ss is not Due to a Splicing Event

The 10 nt sequence is a 5′-splicing donor site and therefore can be recognized by endogenous splicing factors to eventually mediate splicing, providing a 3′-acceptor splicing site is located in the vicinity. Sequence searches (Brendel et al., 2004, Bioinformatics 20(7):1157-1169; Kleffe et al., 1996, Nucl Acids Res 24(23):4709-4718; Brendel et al., 1998, J Mol Biol 276(1):85-104; Brendel and Kleffe, 1998, Nucl Acids Res 26(20):4728-4757; Usuka et al., 2000, J Mol Biol 297(5):1075-1085) indicated that a putative 3′ acceptor site is present within the NOS terminator sequence (FIG. 9A). A possible explanation of down-regulation of gene expression would be a consequence of a splicing event occurring between the 5′ donor site and the 3′-acceptor splicing site within the NOS terminator and generating an aberrant mRNA. RT-PCR was performed using primers annealing downstream of the putative acceptor site (REV2, FIG. 9A). The data reveal that in comparison to GFP, GFP-2×5′ss generates a larger PCR product of about the size of the inserted 2×5′ss SacI fragment, therefore confirming that no splicing event has occurred and indicating that the inserted 5′ donor sites behave as unpaired 5′-donor splice sites.

5′ss-Mediated Inhibition of Expression of an Endogenous Gene

To evaluate the efficacy of the approach for gene knock-down in plants, the effect on the endogenous phytoene desaturase (PDS) was tested. For this purpose, N. benthamiana leaves were agroinfiltrated with the PDS-2×5′ss construct and leaf samples were taken at 3 dpi and 7 dpi. Samples were taken from two separate leaves from three plants per time-point. The effect of PDS-2×5′ss on PDS expression was analysed at the mRNA level by monitoring PDS mRNA level by Real time “Taqman” RT-PCR analysis by amplification of a portion of PDS upstream of the region used in construct PDS-2×5′ss. The PDS mRNA levels were normalised to ubiquitin mRNA in all samples as previously described (Lacomme et al., 2003, Plant J 34:543-553). As presented in FIG. 10, the level of PDS mRNA detected in leaf samples agroinfiltrated with the PDS-2×5′ss construct were significantly lower than those detected in the controls (infiltrated with a different construct or non-infiltrated). The PDS mRNA levels in PDS-2×5′ss were down by 13-fold by 3 dpi and 20-fold by 7 dpi in comparison to control leaves. This demonstrates that 5′ss-mediated inhibition can target efficiently not only co-delivered genes or transgenes, but also endogenous genes such as PDS.

### Example 2

### Materials and Methods

### Plasmid Constructs

Annealed U1-2 pair of primers were ligated into pBI121 plasmid digested by SacI restriction endonuclease giving plasmid pGUS-2×5′ss. The tandem of 5′ss was located in 3′UTR between stop codon of GUS cDNA (upstream) and nopaline synthase terminator (downstream). The same strategy was employed to construct plasmid containing three tandem repeats of 5′ss (6 copies of 5′ss) using instead phosphorylated oligonucleotides for annealing and ligation. A GUS construct harbouring three copies of the 5′ss tandem repeat was generated and the sense orientation of the repeats were confirmed by sequencing. Introduction of spacer sequences into existing pGFP-2×5′ss vector digested with restriction endonuclease AscI was done as follows. A pair of oligonucleotides (5′-cgcgccgatgcagatattcgtaattatgcgggcaacgtctggtatcagcgg-3′ (SEQ ID No: 23) and 5′-cgcgccgctgatacCagacgttgcccgcataattacgaatatctgcatcgg-3′) (SEQ ID No: 24) was annealed and elongated using Taq polymerase. Resulting dsDNA was digested by AscI restriction endonuclease and ligated into pGFP-2×5′ss vector giving pGFP-5′ss-Spacer50-5′ss. A pair of oligonucleotides (5′-aaggcgcgccgatgcagatattc-3′ (SEQ ID No: 25) and 5′-aaggcgcgccgcgcttgctgagtttc-3′ (SEQ ID No: 26)) was used for the generation of a 1000 bp PCR product using GUS coding sequence (pos. 188-1185) as a template. The resulting PCR product was cloned into pGFP-2×5′ss vector giving pGFP-5′ss-Spacer1000-5′ss plasmid.

GUS Assay

Collected leaf discs were homogenized in TissueLyser (Qiagen) in the presence of 0.6 ml homogenization buffer (50 mM Na-Phosphate pH7, 10 mM β-mercaptoethanol, 1 mM EDTA pH8, 0.1% sarcosyl, 0.1% Triton X-100). The extract was spun-down in a microcentrifuge (2000 rpm, 15 min. at 4° C.). Then 0.5 ml of supernatant was transferred into 96-well plates and assayed immediately according to FluorAce 6-glucuronidase Reporter Assay Kit manual (BioRad cat No. 170-3151). The level of GUS expression was measured as activity of β-glucuronidase enzyme per minute per milligram of total protein. Total protein in the samples was measured by Bradford assay.

### Comparison of Level of Gene Expression Down-Regulation of Plasmids Carrying One or Two Unpaired Copies of 5′ss Located in a 3′UTR.

Nicotiana benthamiana leaves were agroinfiltrated with the constructs: pBINmgfp5-ER (GFP), pGFP-5′ss (GFPart1) or pGFP2×5′ss (GFPart2). A relative fluorescence was measured as described in Material and Methods section above. One copy of unpaired 5′ss is able to inhibit gene expression in cis by 30% whereas a construct carrying two copies of unpaired 5′ss down-regulates gene expression by up to 95%. The results are shown in FIG. 11.

Effect of Spacer Length Inserted Between Two 5′ Splicing Donor Sites on Down-Regulation of Gene Expression in cis and trans.

Nicotiana benthamiana leaves were agroinfiltrated with constructs: pBINmgfp5-ER (GFP), pGFP-2×5′ss (GFP-ART), pGFP-5′ss-spacer50-5′ss (Spacer50) or pGFP-5′ss-spacer1000-5′ss (Spacer1000) in presence (white) or absence (black) of GFP construct. Relative fluorescence was measured as described in Material and Methods section above. The data are expressed as Arbitrary Units of Relative fluorescence. No significant difference in GFP downregulation both in cis and in trans were observed in comparison to the original 6 nucleotides spacer present in the GFP-ART construct. The results are shown in FIG. 12.

Influence of Additional Copies of 5′ss within 3′UTR on Expression of GUS in cis.

Construct of β-glucuronidase (GUS) containing 3 copies of a tandem repeat of 5′ss in the 3′UTR (ie. a total of 6 copies of the 5′ss sequence) was prepared and compared to pGUS-2×5′ss. Nicotiana benthamiana leaves were agroinfiltrated with pBI121 (GUS), pGUS-2×5′ss (GUS-ART), pGUS-6×5′ss (GUS-ART(3)) or pGFP-2×5′ss (GFP-ART). To assess the level of GUS cDNA expression β-glucuronidase assay was done. The results were shown as enzymatic activity of β-glucuronidase in relative fluorescence units per minute per milligram of total protein. Activity of GUS expressed from pBI121 plasmid was set as 100%. No significant difference in β-glucuronidase activity was observed between GUS-ART (one copy of the tandem 5′ss sequence) and GUS-ART3x (3 copies of the tandem 5′ss sequence). The results are shown in FIG. 13.

Influence of Additional Copies of 5′ss within 3′-UTR on Expression of GFP in trans.

A construct of β-glucuronidase containing 3 copies of a tandem repeat of 5′ss (ie. 6 copies of the 5′ss sequence) in the 3′-UTR was prepared and compared to pGUS-2×5′ss. Nicotiana benthamiana leaves were co-agroinfiltrated with pBI121 (GUS), pGUS-2×5′ss (GUS-ART), pGUS-6×5′ss (GUS-ART(3)) or pGFP-2×5′ss (GFP-ART) constructs in presence of plasmid pGFP-2×5′ss. Constructs carrying β-glucuronidase (GUS) cDNA contains an identical 3′-UTR (252 nucleotides long) to the construct carrying the GFP cDNA sequence. It was previously shown that 3′-UTR can trigger down-regulation of gene expression in trans by 50-60%. Relative fluorescence was measured as described earlier in Example 1. Additional copies of the 5′ss within the 3′UTR (GUS-ART(3) construct, containing 3 copies of the tandem repeat 5′ss) did not significantly affect the downregulation of gene expression in trans of a co-expressed GFP transgene. Data are expressed in arbitrary units of relative fluorescence emission to a GFP construct. The results are shown in FIG. 14.

5′ss-Mediated Knock Down in Different Plant Species.

Down-regulation of GFP expression in Nicotiana tabacum var Xanthii (Panel a, left) and Nicotiana tabacum var. Samsun (Panel b, right). N.b.: Down-regulation of gene expression in cis by unpaired 5′ss in onion (Allium cepa) epidermal cells previously demonstrated in FIG. 2. The results are shown in FIG. 15.

### Example 3

### Effect of 5′ss Sequence on Expression in Mammalian Cells

### Experimental Procedures

### Plasmid Construction

### Plasmid DNA Transfection of Hela Cells in 24-Well Format Plates

1. Plate 0.5-2×10⁵ cells in 500 μl of growth medium without antibiotics and incubate cells at 37° C. for 24 hours (reach 90-95% confluence).

2. For each transfection sample, prepare mixes as follows:
a. Dilute 150 ng of DNA in 50 μl of Opti-MEM® I Reduced Serum Medium (Gibco) without serum (or other medium without serum). Mix gently.
b. Mix Lipofectamine™ 2000 (Invitrogen) gently before use, then dilute the 1 μl in 50 μl of Opti-MEM® I Medium. Incubate for 5 minutes at room temperature. Note: Proceed to Step c within 25 minutes.
c. After the 5 minute incubation, combine the diluted DNA with diluted Lipofectamine™ 2000 (total volume=100 μl). Mix gently and incubate for 20 minutes at room temperature.

3. Add 100 μl of the transfection mix to each well containing approximately 2.5×10⁵ cells in 5000 medium. Mix gently by rocking the plate back and forth.

4. Incubate cells at 37° C. in a CO₂ incubator for 18-48 hours prior to testing for transgene expression. Medium may be changed after 4-6 hours.

FIG. 16 shows the constructs formed as a schematic representation of pEGFP (pEGFP-C1 expression vector, Clonetech), pEGFPart5 and pDsRED constructs. As indicated above, the plasmids were constructed by inserting tandem 5′-splicing donor sites into construct pEGFP by subcloning into the mammalian expression vector pEGFP using SacI restriction site. The CMV promoter and the SV40 transcriptional terminator are represented.

Protein Extraction from Hela Cells
1. Remove growth medium from the cells by decantation or aspiration.
2. Wash cells to remove residual medium. Slowly add a volume of PBS, equal to the original medium volume being careful not to dislodge cells. Mix gently and remove the wash solution. Repeat the wash once in order to remove any other minor contaminants.
3. After removal of the final wash solution from the cells, add 100 ul of RIPA Buffer (Pierce) into each of 24 well (1 ml for 0.5 to 5×10⁷ cells). Incubate on ice or in a refrigerator (2-8° C.) for five minutes.
4. Rapidly scrape the plate to remove and lyse residual cells. Transfer the cell lysate to a tube on ice. The lysate can either be used immediately or flash-frozen in liquid nitrogen and stored at −70° C. for future use. It is best to freeze the lysate before clarification, since the freeze-thaw cycle may cause some denatured protein to aggregate.
5. Clarify the lysate by centrifugation at 8,000 g for 10 minutes at 4° C. to pellet the cell debris. Note: If a mucoid aggregate of denatured nucleic acids is present, carefully remove it with a micropipette before centrifugation.
6. Carefully transfer the supernatant containing the soluble protein to a tube on ice for further analysis.
7. Total protein concentration was quantified using Bradford assay. PAGE and immunoblotting techniques were as previously described.

### Assessment of EGFP Expression in HeLa Cells Using Epifluorescence Microscopy and Western Blotting Techniques.

Experiments were effectuated in 24-wells plate format. In each well approximately 2.5×10⁵ HeLa cells were grown. Cells were co-transfected with the following constructs combinations: pEGFP+pDsRED, pEGFPart5+pDsRED, 2×pEGFP+pDsRED, pEGFP+pEGFPart5+pDsRED (See FIG. 16) and enumeration of cells expressing EGFP was effectuated using a fluorescence microscope (Nikon Optiphot epifluorescence microscope) and 3CCD Color Video Camera (KY-F558 Photonic Science). Approximately 1% of HeLa cells, transiently transfected with pEGFPart5 construct displayed green fluorescence, whereas about 60% of cells transfected with pEGFP construct showed fluorescence. Two days post transfection pictures were taken using a Nikon Optiphot epifluorescence microscope using either a green or a red filter to detect EGFP and DsRED fluorescence respectively (see FIG. 17). The cells transfected with the mix pEGFP+pEGFPart5+pDsRED showed similar number of EGFP fluorescent cells as for cells transfected with pEGFP+pDsRED constructs (FIG. 17).

Bright field and fluorescent (green and red) merged images are presented. Proliferating HeLa cells expressing EGFP appears bright green under the fluorescence microscope, the bright field image allow the observation of non-expressing cells (especially with constructs pEGFPart5+pDsRED). Note that due to the slowest maturation process of DsRED in comparison to EGFP the red fluorescence is barely detectable at 2 days post transfection as opposed to the green fluorescence.

Western blot analysis of the cells transfected with the above-mentioned construct combinations was effectuated (see FIG. 18). A significantly lower accumulation of EGFP was observed when cells were transfected with the construct pEGFPart5 harbouring the tandem 5′ss sequence for the samples pEGFPart5+pDsRED and pEGFP+pEGFPart5+pDsRED as opposed to control samples pEGFP+pDsRED and 2×pEGFP+pDsRED respectively. The DsRED is used as a marker of transfection and internal calibrator for western blot analysis.

Construct pEGFPart5 was engineered to harbour a tandem insertion of 5′ss in the 3′ UTR of EGFP. Their transient expression in HeLa cells causes a knock-down of GFP expression in cis (line A2) but also in trans (line A4) in comparison to control pEGFP constructs (A1 and A3). Western blot analysis shows accumulation of green fluorescence protein (panel A) or co-expressed red fluorescence protein (panel B); line 1: pEGFP+pDsRED; line 2: pEGFPart5+pDsRED; line 3: 2×pEGFP+pDsRED; line 4: pEGFP+pEGFPart5+pDsRED. The blots were probed with antibodies against green fluorescent protein (GFP) (panel A) or against red fluorescent protein (RFP) used as an internal calibrator (panel B).

These data suggests that the pEGFPart5 construct express EGFP to significantly lower levels in HeLa cells both in cis and in trans by affecting expression of the modified pEGFPart5 construct and a distinct pEGFP construct.

Standard exemplary method for transfection of mammalian cells

### Cell and DNA Preparation:

1. Plate cells 24 hours before transfection. Usual plating density is 1×10⁶ cells/100 mm dish/10 mls complete media.
   Note: If transfecting a suspension culture, suspension cell concentration should be 5×10⁶/ml. Suspension cells grown in RPMI may be difficult to transfect with this kit. It is recommended to use a DMEM for suspension transfection in this case.
2. Feed cells fresh, complete media 3 hours before transfection.
3. All DNAs used should be phenol, phenol/chloroform/isoamyl alcohol extracted ethanol precipitated and dissolved in sterile UltraPure water or a tris/EDTA solution.

### Transfection Procedure:

1. 1 ml of calcium phosphate precipitate is needed for each 100 mm plate of cells to be transfected.
2. Prepare 1×HBS fresh for each experiment. 0.5 ml of 1×HBS is needed for each 100 mm plate.
3. Formula for 1×HBS is as follows:
   Add 0.88 ml sterile UltraPure water to tube
   Add 0.1 ml 10×HBS and mix well
   Add 150 NaOH Solution and mix well
   (note: the pH will be correct and need not be checked)

### Formula for 1 ml Calcium Phosphate DNA Precipitate:

1. Set up two sterile polypropylene tubes for each DNA to be precipitated, label tubes #1 and #2 along with the DNA to be used
2. Add to tube #1: 0.5 mls of 1×HBS and 100 of phosphate solution.
3. Add to tube #2: 0.43 mls of UltraPure water minus volume of DNA. Total DNA should equal 20 μg.
   Note: If genomic DNA is being used, the total DNA should equal 30 μg. Genomic DNA will replace carrier and plasmid DNA's.
4. Gently mix the DNAs into the water.
5. Add 600 of calcium solution and mix gently

### Forming the Calcium Phosphate and DNA Precipitate:

1. Place a sterile 1 ml pipet into tube #1 and gently bubble air through the solution so that it is slowly mixing.
2. Draw the contents of tube #2 up into an appropriately sized sterile pipet. Add slowly, dropwise, to the gently bubbling and mixing solution in tube #1. As the two solutions mix they will appear milky and then form a white precipitate. Continue to bubble and add slowly until the entire contents of tube #2 have been added.
3. Allow the suspension to sit at room temperature for 20 minutes before adding to the cells.

### Adding the Precipitate to the Cells:

1. Mix the precipitate well by pipeting or vortexing, making sure that any large clumps that may have formed on the bottom of the tube are broken up and that the precipitate is evenly resuspended.
2. Add 1 ml of suspension to a 100 mm plate containing 10 mls of complete media. The suspension must be added slowly, dropwise, while gently rocking the media in the plate.
3. Return the plates to the incubator and leave the precipitate on for 12-24 hours.

### Maintenance of Transfected Cells:

1. Remove the media containing the precipitate and add fresh complete media leaving this media on for 24 hours.
2. Remove the media and add the appropriate selection media to select stable colonies or add complete media for transient expression incubation.

1. A polynucleotide comprising a gene having an exon containing a stop codon and said polynucleotide having a transcription terminator for said gene, wherein the gene has a sequence derived from a 5′ splicing site located upstream of the transcription terminator and within the exon of the gene containing the stop codon.

2. The polynucleotide as claimed in claim 1 wherein said gene encodes a protein or a portion thereof.

3. (canceled)

4. The polynucleotide as claimed in claim 1 wherein said gene is in cDNA form.

5. The polynucleotide as claimed in claim 1 wherein the 5′ splicing site sequence is located downstream of the stop codon.

6. The polynucleotide as claimed in claim 1 wherein the 5′ splicing site sequence is located upstream of the stop codon.

7. The polynucleotide as claimed in claim 1 wherein the 5′ splicing site sequence is located within 20 to 1000 nucleotides upstream of the transcription terminator.

8. (canceled)

9. The polynucleotide as claimed in claim 1 wherein two or more copies of the 5′ splicing site sequence are present.

10. The polynucleotide as claimed in claim 9 wherein the two or more copies of the 5′ splicing site sequence are present in tandem.

11. The polynucleotide as claimed in claim 1 wherein the 5′ splicing site sequence is the sequence 5′-MAGGTRAGTA-3′ (SEQ ID NO: 1) where M=A or C and R=A or G, or a variant thereof in which 1, 2, 3 or 4 nucleotides are substituted or deleted.

12. The polynucleotide as claimed in claim 11 wherein the 5′ splicing site sequence is the sequence 5′-CAGGTAAGTA-3′ (SEQ ID NO: 2) or a variant thereof in which 1, 2, 3 or 4 nucleotides are substituted or deleted.

13. (canceled)

14. (canceled)

15. (canceled)

16. A polynucleotide vector having a 5′ splicing site sequence located upstream of a transcription terminator wherein two or more copies of the 5′ splicing site sequence are present in tandem.

17. A polynucleotide vector having a 5′ splicing site sequence located upstream of a transcription terminator, wherein said vector comprises at least a portion of an open reading frame containing a stop codon or at least a portion of a 3′UTR, wherein said 5′ splicing site sequence is located in the 3′UTR or open reading frame.

18. The vector as claimed in claim 16 wherein said vector comprises at least a portion of a 3′ UTR of a target gene, wherein said 3′ UTR portion comprises at least one copy of the 5′ splicing site sequence.

19. The vector as claimed in claim 16 wherein said vector comprises at least a portion of an open reading frame of a target gene, wherein said open reading frame contains a stop codon, wherein said open reading frame comprises at least one copy of the 5′ splicing site sequence.

20. The vector as claimed in claim 16 wherein said vector is able to insert said 5′ splicing site sequence within the 3′UTR of a target gene.

21. The vector as claimed in claim 16 wherein said vector is able to insert said 5′ splicing site sequence within an open reading frame of a target gene, said open reading frame containing a stop codon.

22. The vector as claimed claim 16 wherein the 5′ splicing site sequence is located within 20 to 1000 nucleotides upstream of a transcription terminator.

23. The vector as claimed in claim 16 wherein the 5′ splicing site sequence is the sequence 5′-MAGGTRAGTA-3′ (SEQ ID NO: 1) where M=A or C and R=A or G, or a variant thereof in which 1, 2, 3 or 4 nucleotides are substituted or deleted.

24. The vector as claimed in claim 23 wherein the 5′ splicing site sequence is the sequence 5′-CAGGTAAGTA-3′ (SEQ ID NO: 2) or a variant thereof in which 1, 2, 3 or 4 nucleotides are substituted or deleted.

25. The vector as claimed in claim 23 which further includes a polyadenylation signal.

26. The vector as claimed in claim 25 wherein the polyadenylation signal is AAUAAA.

27. The vector as claimed in 16 wherein said vector includes a polynucleotide sequence of a target gene in a form suitable for expression.

28. The vector as claimed in claim 27 which includes a full length sequence of the target gene.

29. (canceled)

30. The vector as claimed in claim 27 wherein the target gene is a chimeric gene.

31. The vector as claimed in claim 26 wherein said vector comprises at least a portion of a 3′ UTR of a target gene, wherein said 3′ UTR portion comprises at least one copy of the 5′ splicing site sequence.

32. (canceled)

33. (canceled)

34. (canceled)

35. A method of reducing expression of a target gene wherein said gene comprises at least a portion of either an open reading frame containing a stop codon or of a 3′UTR, said method comprising modifying the portion of open reading frame or 3′UTR by inserting a 5′ splicing site sequence therein and upstream of a transcription terminator of said target gene.

36. The method of claim 34 wherein said 5′ splicing site sequence is inserted into the open reading frame of said gene.

37. A method of reducing expression of a target gene, said method comprising providing a vector comprising, in functional relationship, a transcription initiator, a targeting sequence and a transcription terminator, said vector further comprising a 5′ splicing site sequence upstream of said transcription terminator, and wherein 21 nucleotides of said targeting sequence has at least a 95% sequence identity to 21 nucleotides of the target gene.

38. The method as claimed in claim 37 wherein 21 nucleotides of the targeting sequence has 100% sequence identity to 21 nucleotides of the target gene.

39. The method as claimed in claim 37 wherein said targeted sequence is in an open reading frame of the target gene.

40. The method as claimed in claim 37 wherein said targeted sequence in a 3′ UTR of the target gene.

41. A host cell containing a polynucleotide of claim 1.

42. A host cell containing a polynucleotide vector of claim 16.

43. (canceled)

44. (canceled)

45. (canceled)

46. (canceled)

47. (canceled)

48. (canceled)

49. The vector as claimed in claim 24 which further includes a polyadenylation signal.

50. The vector as claimed in claim 17 wherein said vector includes a polynucleotide sequence of a target gene in a form suitable for expression.


SEQUENCE LISTING (26 sequences)

SEQ ID NO: 1 - DNA, 10 bp, Artificial Sequence
  Synthetic embodiment of 5′ splicing site sequence
maggtragta

SEQ ID NO: 2 - DNA, 10 bp, Artificial
  Synthetic embodiment of 5′ splicing site sequence
caggtaagta

SEQ ID NO: 3 - DNA, 308 bp, Artificial
  Synthetic embodiment of nucleotidic composition of the 3′ UTR of GFP-2x5′ splicing site
taagagctcgagcaggtaagtaggcgcgcccaggtaagtagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 4 - DNA, 855 bp, Artificial
  Synthetic embodiment of nucleotidic composition of the 3′ UTR of GFP-MCS-2x5′ splicing site-OCS
taaccctaggtacgtaggcgcgccattataaatctagacaggtaagtacggatccgcaggtaagtagacgtcctctagcttaattaagacccgggactagtccctagagtcctgctttaatgagatatgcgagacgcctatgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctgagcagga

SEQ ID NO: 5 - DNA, 20 bp, Artificial
  Forward primer used for semi-quantitative RT-PCR of GFP expression
gggcacaaattttctgtcag

SEQ ID NO: 6 - DNA, 24 bp, Artificial
  Reverse primer used for semi-quantitative RT-PCR of GFP expression
gttgtgggagttgtagttgtattc

SEQ ID NO: 7 - DNA, 31 bp, Artificial
  REV1 primer
aaataacgtcatgcattacatgttaattatt

SEQ ID NO: 8 - DNA, 27 bp, Artificial
  REV2 primer
ttctatcgcgtattaaatgtataattg

SEQ ID NO: 9 - DNA, 20 bp, Artificial
  forward primer
tccacacaatctgccctttc

SEQ ID NO: 10 - DNA, 27 bp, Artificial
  reverse primer
gcgagctccgcggcctttttttttttt

SEQ ID NO: 11 - DNA, 30 bp, Artificial
  Primer for amplification of CaMV 35s::mgfp5-ER::3′-NOS derived from pBINmgfp5-ER plasmid
cccaagctttttcagaaagaatgctaaccc

SEQ ID NO: 12 - DNA, 32 bp, Artificial
  Primer for amplification of CaMV 35s::mgfp5-ER::3′-NOS derived from pBINmgfp5-ER plasmid
cccaagcttgatctagtaacatagatgacacc

SEQ ID NO: 13 - DNA, 27 bp, Artificial
  self annealing primer
cgagmaggtragtaggcgcgccgagct

SEQ ID NO: 14 - DNA, 27 bp, Artificial
  self annealing primer
cggcgcgcctacttacctgctcgagct

SEQ ID NO: 15 - DNA, 37 bp, Artificial
  self annealing primer
cgagmaggtragtaggcgcgccmaggtragtagagct

SEQ ID NO: 16 - DNA, 37 bp, Artificial
  self annealing primer
ctacttacctgggcgcgcctacttacctgctcgagct

SEQ ID NO: 17 - DNA, 45 bp, Artificial
  Primer used for amplification of GFP ORF from pBINmgfp5-ER plasmid
gtgtgtctcgagccatggccaagactaatctttttctctttctca

SEQ ID NO: 18 - DNA, 57 bp, Artificial
  Primer used for amplification of GFP ORF from pBINmgfp5-ER plasmid
gtgtgtggcgcgcctacgtacctagggttaaccaagctcatcatgtttgtatagttc

SEQ ID NO: 19 - DNA, 62 bp, Artificial
  Annealed primer used for ligating ChSA intron sequences
cgcgccattataaatctagacaggtaagtacggatccgcaggtaagtagacgtcctctagcc

SEQ ID NO: 20 - DNA, 62 bp, Artificial
  Annealed primer for ligating ChSA intron sequences
ccggggctagaggacgtctacttacctgcggatccgtacttacctgtctagatttataatgg

SEQ ID NO: 21 - DNA, 33 bp, Artificial
  Forward primer for amplification of 314 bp PDS cDNA fragment
atgggatccatgaaggaactagcgaagcttttc

SEQ ID NO: 22 - DNA, 35 bp, Artificial
  Reverse primer for amplification of 314 bp PDS cDNA fragment
tacgagctcttagttcactatgctaactacgcttg

SEQ ID NO: 23 - DNA, 51 bp, Artificial
  oligonucleotide used for creation of spacer sequences for pGFP-2x5′ splicing site vector
cgcgccgatgcagatattcgtaattatgcgggcaacgtctggtatcagcgg

SEQ ID NO: 24 - DNA, 51 bp, Artificial
  oligonucleotide used for creation of spacer sequences for pGFP-2x5′ splicing site vector
cgcgccgctgataccagacgttgcccgcataattacgaatatctgcatcgg

SEQ ID NO: 25 - DNA, 23 bp, Artificial
  Primer 1 for GUS amplification (1000 bp PCR product)
aaggcgcgccgatgcagatattc

SEQ ID NO: 26 - DNA, 26 bp, Artificial
  Primer 2 for GUS amplification (1000 bp PCR product)
aaggcgcgccgcgcttgctgagtttc